(19) United States
(12) Patent Application Publication
(10) Pub. No.: US 2008/0031854 A1
(43) Pub. Date: Feb. 7, 2008

(54) THIOLATED MACROMOLECULES AND METHODS OF MAKING AND USING THEREOF

(76) Inventors: Glenn D. Prestwich, Salt Lake City, UT (US); Monica Serban, Salt Lake City, UT (US)

Correspondence Address: HELLER EHRMAN LLP, 275 MIDDLEFIELD ROAD, MENLO PARK, CA, 94025-3506, US

(21) Appl. No.: 11/776,519

(22) Filed: Jul. 11, 2007

Related U.S. Application Data
(60) Provisional application No. 60/806,965, filed on Jul. 11, 2006.

(51) Int. Cl. A61K 31/7008; A01N 1/02; A61K 45/00; A61P 17/02; A61P 41/00; C07H 15/00; C07H 5/04; C07K 14/00; C12N 5/06

(52) U.S. Cl. 424/93.1; 435/1.1; 435/325; 514/25; 530/350; 536/17.6; 536/55.2

(57) ABSTRACT

Described herein are thiolated macromolecules and methods of making and using thereof.

### RELATED APPLICATIONS

This application claims the benefit of U.S. Provisional Application No. 60/806,965 filed Jul. 11, 2006.

### BACKGROUND

Arthritis is used to generically refer to over one hundred pathological conditions that cause joint pain and inflammation. The two most common diseases responsible for the aforementioned symptoms are osteoarthritis (OA) and rheumatoid arthritis (RA). Osteoarthritis (OA), also known as degenerative arthritis, is caused by the wear and tear of the joints and affects over 20 million people in the United States. OA affects particularly large weight-bearing, synovial joints. In contrast, RA is an autoimmune disease that causes inflammation and ultimately results in the destruction of cartilage and bone. Anatomically, a synovial joint features a synovial membrane, cartilage, subchondral bone, synovial fluid and a joint capsule. In arthritis, the articular cartilage slowly degrades and ultimately disappears. However, changes also occur in the subchondral bone, the joint capsule and in the synovial fluid. High molecular weight hyaluronic acid (HA) is a major component synovial fluid. Conversely, in the synovial fluid of OA patients, the HA concentration is lower than normal, and the molecular weight distribution is shifted to lower average mass.

HA oligosaccharides or HA hexasaccharides were also found to induce nitric oxide synthase leading to increased production of nitric oxide in bovine articular chondrocytes (cartilage forming cells). In cultures of human normal adult chondrocytes, HA oligosaccharide treatment led to the loss of proteoglycan (one of the extracellular matrix components) by induction of matrix metalloproteinase 13, through activation of NFkappaB and p38 MAP kinase. Bovine articular chondrocytes were shown to undergo a dose-dependent chondrolysis when treated with HA oligosaccharides. All these processes are associated with the progression and aggravation of arthritis.

Viscosupplementation is an intra-articular treatment option for arthritis that is targeted to restore the physiological viscoelasticity of the synovial fluid. Viscosupplementation involves the injection of high molecular weight HA directly into the arthritis affected joint. However, the poor biomechanical properties and rapid biodegradation of natural HA suggests that chemically-modified HA derivatives with longer in vivo residence times would yield better clinical outcomes.

The use of thiolated macromolecules in pharmaceutical applications has received considerable attention. For example, thiols can be used to reduce or prevent free radicals or reactive oxygen species from causing cell damage or death. Free radicals and reactive oxygen species can cause severe pain and inflammation in a subject. In other applications, two or more thiolated macromolecules can be coupled to produce new macromolecule scaffolds with multiple activities including wound healing and drug delivery. Described herein are thiolated macromolecules and methods for making and using the same.

We provide herein experimental data that indicate that HASH may have utility for arthritis treatment. The material is HA-based, which would provide biocompatibility, is well tolerated by cells and showed promising results in a rat arthritis pilot study. The presence of the SH groups of HASH may act as radical scavengers, thus protecting cells from the damaging effects of reactive oxygen species. Because of the HA scaffold, it can also serve as a joint lubricant, thus encompassing a dual protective function. The macromolecule is not readily crosslinkable via previously employed chemical crosslinking techniques. However, if needed, its structure could further be chemically crosslinked via other crosslinking strategies (i.e., divinyl sulfone or intra-molecular esterification crosslinking).

### SUMMARY OF THE INVENTION

Described herein are thiolated macromolecules and methods of making and using thereof. More specifically, described herein is the chemical synthesis and characterization of a novel thiol containing HA derivative wherein the material obtained is not suitable for hydrogel formation via crosslinking. As the macromolecule yields viscous solutions when dissolved in water, this property makes it suitable for viscosupplementation-type applications as protective against oxidative stress and diseases.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1. Synthetic scheme and structure of HASH

FIG. 2. ¹H-NMR spectra in D₂O. Panel A, HA; Panel B, HASH

FIG. 3. GPC analysis (UV) of HASH. The depression in the base line at approximately 21 min is due to the water used to dissolve the sample.

FIG. 4. SAMSA fluorescein derivatization of HASH. Panel A, Structure of SAMSA fluorescein; Panel B, A₄₉₄ absorbance of SAMSA fluorescein derivatized HASH (*** p<0.005, versus the HA control). Columns represent mean ± S.D., n=4. Inset—fluorescence intensities of SAMSA derivatized solutions under UV light (254 nm).

FIG. 5. Reaction of HASH with sodium 4-(hydroxymercuri)benzoate. Panel A, Reaction scheme; Panel B, ¹H-NMR spectrum of HASH-mercuribenzoate adduct

FIG. 6. Reaction of HASH with sodium iodoacetate. Panel A, Reaction scheme; Panel B, ¹H-NMR spectrum of S-carboxymethyl HASH

FIG. 7. Proliferation of T31 fibroblasts as determined by MTS assay. Panel A, with added 120 kDa HA (white) or 200 kDa HA (gray bars); Panel B, with added HASH. Each column represents the mean ± S.D., n=6 (*** p<0.005, ** p<0.05 and * p>0.05 versus the control group).

FIG. 8. Primary ovine chondrocyte apoptosis rates induced by H₂O₂. Panel A, HA-treated chondrocytes; Panel B, HASH-treated chondrocytes (*** p<0.005, ** p<0.05 and * p>0.05 versus the H₂O₂-only treated control group).

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

References in the specification and concluding claims to parts by weight, of a particular element or component in a composition or article, denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. For example, a polysaccharide that contains at least one —OH group can be represented by the formula Y—OH, where Y is the remainder (i.e., residue) of the polysaccharide molecule.

Variables such as R¹-R¹⁰, A¹, A², A′, G, M, U, V, X, X′, Y, Y′, and Z used throughout the application are the same variables as previously defined unless stated to the contrary.

The term “alkyl group” as used herein is a branched or unbranched saturated hydrocarbon group of 1 to 24 carbon atoms (e.g. C₁₋₂₄ alkyl), such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “perfluoroalkyl group” or “fluoroalkyl” as used herein means a branched or unbranched saturated hydrocarbon group of 1 to 24 carbon atoms, wherein at least one of the hydrogen atoms is substituted with fluorine. A perfluoroalkyl group may also mean that all hydrogen atoms of the alkyl group are substituted with fluorine.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “halogen” as used herein is fluoride, chloride, bromide, or iodide.

The term “leaving group” means a group that may be readily displaced by a nucleophile that has a greater affinity for the carbon atom to which the leaving group is attached to than the leaving group. Examples of such leaving groups include halo (bromo, chloro and iodo) and organosulfonyloxy groups. Particularly preferred organosulfonyloxy groups include alkylsulfonyloxy and arylsulfonyloxy groups are methanesulfonyloxy, benzenesulfonyloxy, p-toluenesulfonyloxy, p-nitrobenzenesulfonyloxy or m-nitrobenzenesulfonyloxy.

The term “polyalkylene group” as used herein is a group having two or more CH₂ groups linked to one another. The polyalkylene group can be represented by the formula —(CH₂)ₙ—, where n is an integer of from 2 to 25.

The term “polyether group” as used herein is a group having the formula —[(CHR)ₙO]ₘ—, where R is hydrogen or a lower alkyl group, n is an integer of from 1 to 20, and m is an integer of from 1 to 100. Examples of polyether groups include, polyethylene oxide, polypropylene oxide, and polybutylene oxide.

The term “polythioether group” as used herein is a group having the formula —[(CHR)ₙS]ₘ—, where R is hydrogen or a lower alkyl group, n is an integer of from 1 to 20, and m is an integer of from 1 to 100.

The term “polyimino group” as used herein is a group having the formula —[(CHR)ₙNR]ₘ—, where each R is, independently, hydrogen or a lower alkyl group, n is an integer of from 1 to 20, and m is an integer of from 1 to 100.

The term “polyester group” as used herein is a group that is produced by the reaction between a compound having at least two carboxylic acid groups with a compound having at least two hydroxyl groups.

The term “polyamide group” as used herein is a group that is produced by the reaction between a compound having at least two carboxylic acid groups with a compound having at least two unsubstituted or monosubstituted amino groups.

I. Thiolated Macromolecules and Preparation Thereof

Described herein are thiolated macromolecules. In one aspect, the crosslinker comprises the formula I
Y—X—R—SH I
wherein
Y is a residue of a macromolecule;
X is —O—, —S—, —NH—, or NR′—;
R′ is C₁₋₅ alkyl; and
R is a substituted or unsubstituted C₂ or C₃ alkylene group.
In a particular variation of the formula I, X is a residue of a nucleophilic group.

The macromolecule is any compound having at least one nucleophilic group. Examples of nucleophilic groups include, but are not limited to, hydroxyl, thiol, and substituted or unsubstituted groups. Referring to formula I, X is the residue of the nucleophilic group of the macromolecule. In one aspect, a nucleophilic group is capable of reacting with a strained heterocycloalkyl group and ring-open the group. In another aspect, X is O or NH. In the case when the nucleophilic groups is a hydroxyl or amino groups, the hydroxyl or amino group is a free hydroxyl or amino group or it is derived from a carboxylic acid or amide, respectively. In one aspect, the macromolecule is an oligonucleotide, a nucleic acid or a metabolically stabilized analogue thereof, a polypeptide, a glycoprotein, or a glycolipid. In another aspect, the macromolecule is a polysaccharide or a protein.

Polysaccharides useful in the methods described herein have at least one nucleophilic group such as, for example, a hydroxyl group. In one aspect, the polysaccharide is a glycosaminoglycan (GAG). Glycosaminoglycans can be sulfated or non-sulfated. A GAG is one molecule with many alternating subunits. For example, HA is (GlcNAc-GlcUA-)x. Other GAGs are sulfated at different sugars. Generically, GAGs are represented by the formula A-B-A-B-A-B, where A is an uronic acid and B is an aminosugar that is either O- or N-sulfated, where the A and B units can be heterogeneous with respect to epimeric content or sulfation. Any natural or synthetic polymer containing uronic acid can be used. In one aspect, Y in formula I is a sulfated-GAG.

There are many different types of GAGs, having commonly understood structures, which, for example, are within the disclosed compositions, such as chondroitin sulfate, dermatan, heparan, heparin, dermatan sulfate, and heparan sulfate. Any GAG known in the art can be used in any of the methods described herein. Alginic acid, pectin, chitosan, and carboxymethylcellulose are among other polysaccharides useful in the methods described herein.

In another aspect, the polysaccharide Y in formula I is hyaluronan (HA). HA is a non-sulfated GAG. Hyaluronan is a well-known, naturally occurring, water soluble polysaccharide composed of two alternatively linked sugars, D-glucuronic acid and N-acetylglucosamine. The polymer is hydrophilic and highly viscous in aqueous solution at relatively low solute concentrations. It often occurs naturally as the sodium salt, sodium hyaluronate. Methods of preparing commercially available hyaluronan and salts thereof are well known. Hyaluronan can be purchased from Seikagaku Company, Novozymes Biopolymer, Novomatrix, Pharmacia Inc., Sigma Inc., and many other suppliers. For high molecular weight hyaluronan it is often in the range of 100 to 10,000 disaccharide units. In another aspect, the lower limit of the molecular weight of the hyaluronan is from 10,000, 20,000, 30,000, 40,000, 50,000, 60,000, 70,000, 80,000, 90,000, or 100,000, and the upper limit is 200,000, 300,000, 400,000, 500,000, 600,000, 700,000, 800,000, 900,000, or 1,000,000, where any of the lower limits can be combined with any of the upper limits.

In one aspect, Y in formula I can also be a synthetic polymer. The synthetic polymer has at least one nucleophilic group. In one aspect, the synthetic polymer residue in formula I comprises polyvinyl alcohol, polyethyleneimine, polyethylene glycol, polypropylene glycol, a polyol, a polyamine, a triblock polymer of polypropylene oxide-polyethylene oxide-polypropylene oxide, a star polymer of polyethylene glycol, or a dendrimer of polyethylene glycol.

In another aspect, Y in formula I is a protein. Proteins useful herein include, but are not limited to, an extracellular matrix protein, a chemically-modified extracellular matrix protein, or a partially hydrolyzed derivative of an extracellular matrix protein. The proteins may be naturally occurring or recombinant polypeptides possessing a cell interactive domain. The protein can also be a mixture of proteins, where one or more of the proteins are modified. Specific examples of proteins include, but are not limited to, collagen, elastin, decorin, laminin, or fibronectin. In one aspect, the protein comprises genetically engineered proteins with additional thiol groups (e.g., cysteine residues). In a further aspect, the protein comprises a synthetic polypeptide that can be a branched (e.g., a dendrimer) or linear with additional thiol groups (e.g., cysteine residues).

In one aspect, Y comprises a residue of a glycosaminoglycan, where the glycosaminoglycan can be sulfated or non-sulfated. In another aspect, Y comprises a residue of hyaluronan. In a further aspect, Y comprises a residue of an N-acetyl-glucosamine, wherein at least one primary C-6 hydroxyl group of the N-acetyl-glucosamine residue is substituted with the group —RSH. Further to this aspect, at least one secondary hydroxyl group is substituted with the group —RSH as well. In another aspect, one primary C-6 hydroxyl group of the N-acetyl-glucosamine residue to about 100% of the primary C-6 hydroxyl groups of the N-acetyl-glucosamine residue are substituted with the group —RSH.

In another aspect, R in formula I is CH₂CH₂, CH₂CH₂CH₂, CH₂CHR⁵, CHR⁵CHR⁵, C(R⁵)₂CHR⁵, or C(R⁵)₂C(R⁵)₂, wherein R⁵ is an alkyl group. In one aspect, Y in formula I is a residue of a hyaluronan, wherein at least one hydroxyl group is substituted with —CH₂CH₂SH.

The compounds having the formula I can be synthesized by the methods described herein. In one aspect, the method comprises reacting a macromolecule comprising at least one nucleophilic group (e.g., hydroxyl group or amino group) with a compound comprising the formula XVII 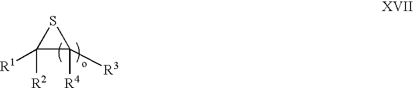
wherein R¹, R², R³, and R⁴ are, independently, hydrogen, an alkyl group, a perfluoroalkyl group, an aryl group, or a heteroaryl group, and o is 1 or 2.

In one aspect, o in formula XVII is 1. In another aspect, o in formula XVII is 1 and R¹-R⁴ are hydrogen. In another aspect, the compound having the formula I is formed by the reaction product between hyaluronan and a compound having the formula XVII, where o is 1 and R¹-R⁴ are hydrogen.

The reaction between the macromolecule and the compound having the formula XVII can be conducted at various reaction temperatures and times, which will vary depending upon the selection of starting materials. The selection of solvents will also vary on the solubility of the starting materials. In certain aspects, it is desirable to conduct the reaction at a pH greater than 7. For example, when the macromolecule has one or more hydroxyl groups, a basic medium may be desired to deprotonate a certain number of the hydroxyl groups and facilitate the reaction between the macromolecule and the compound having the formula XVII.

Any of the compounds described herein can be the pharmaceutically-acceptable salt or ester thereof. In one aspect, pharmaceutically-acceptable salts are prepared by treating the free acid with an appropriate amount of a pharmaceutically-acceptable base. Representative pharmaceutically-acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

In another aspect, if the compound possesses a basic group, it can be protonated with an acid such as, for example, HCl, HBr, or H₂SO₄, to produce the cationic salt. In one aspect, the reaction of the compound with the acid or base is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

Ester derivatives are typically prepared as precursors to the acid form of the compounds. Generally, these derivatives will be lower alkyl esters such as methyl, ethyl, and the like. Amide derivatives —(CO)NH₂, —(CO)NHR and —(CO)NR₂, where R is an alkyl group defined above, can be prepared by reaction of the carboxylic acid-containing compound with ammonia or a substituted amine.

II. Crosslinking Via Oxidative Coupling

In one aspect described herein is a method for coupling two or more thiolated compounds, comprising reacting a first thiolated compound comprising the formula I with a second thiolated compound having at least one SH group in the presence of an oxidant, wherein the first thiolated compound and second thiolated compound are the same or different compounds.

The second thiolated compound is any compound having at least one thiol group. The first and second thiolated compounds can be the same or different compounds. In one aspect, the second thiolated compound can be any macromolecule described above having at least one SH group. In one aspect, the second thiolated compound is a polysaccharide having at least one SH group. Any of the polysaccharides described above can be used as the second thiolated compound. In another aspect, the second thiolated compound comprises a glycosaminoglycan (sulfated or non-sulfated). In a further aspect, the second thiolated compound includes chondroitin sulfate, dermatan, heparan, heparin, dermatan sulfate, heparan sulfate, alginic acid, chitosan, pectin, carboxymethylcellulose, or hyaluronan having at least one SH group.

In one aspect, the second thiolated compound can be a protein having at least one thiol group. In this aspect, the protein can be naturally occurring or synthetic. In one aspect, the protein comprises an extracellular matrix protein or a chemically-modified extracellular matrix protein. In another aspect, the protein comprises collagen, elastin, decorin, laminin, or fibronectin. In one aspect, the protein comprises genetically engineered proteins with additional thiol groups (e.g., cysteine residues). In a further aspect, the protein comprises a synthetic polypeptide that can be a branched (e.g., a dendrimer) or linear with additional thiol groups (e.g., cysteine residues).

The reaction between the first and second thiolated compounds is performed in the presence of an oxidant. In one aspect, the reaction between the first and second thiolated compounds can be conducted in the presence of any gas that contains oxygen. In one aspect, the oxidant is air (e.g., from 0.1 to 100% oxygen). In another aspect, the oxidant can be in an aqueous solution. This aspect also contemplates the addition of a second oxidant to expedite the reaction. In another aspect, the reaction can be performed under an inert atmosphere (i.e., oxygen free), and an oxidant is added to the reaction. Examples of oxidants useful in this method include, but are not limited to, molecular iodine, hydrogen peroxide, alkyl hydroperoxides, peroxy acids, dialkyl sulfoxides, high valent metals such as Co⁺³ and Ce⁺⁴, metal oxides of manganese, lead, and chromium, and halogen transfer agents. The oxidants disclosed in Capozzi, G.; Modena, G. In The Chemistry of the Thiol Group Part II; Patai, S., Ed.; Wiley: New York, 1974; pp 785-839, which is incorporated by reference in its entirety, are useful in the methods described herein.

The reaction between the first and second thiolated compounds can be conducted in a buffer solution that is slightly basic. The amount of the first thiolated compound relative the amount of the second thiolated compound can vary. In one aspect, the volume ratio of the first thiolated compound to the second thiolated compound is from 99:1, 90:10, 80:20, 70:30, 60:40, 50:50, 40:60, 30:70, 20:80, 10:90, or 1:99. In one aspect, the first and second thiolated compounds react in air and are allowed to dry at room temperature. In this aspect, the dried material can be exposed to a second oxidant, such as hydrogen peroxide. The resultant compound can then be rinsed with water to remove any unreacted first and/or second thiolated compound and any unused oxidant. One advantage of preparing coupled compound via the oxidative coupling methodology described herein is that crosslinking can occur in an aqueous media under physiologically benign conditions without the necessity of additional crosslinking reagents.

The compounds produced using the methods described above have at least one fragment comprising the formula VI 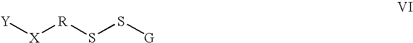
wherein
Y is a residue of a first macromolecule selected from the group consisting of an oligonucleotide, a nucleic acid or a metabolically stabilized analogue thereof, a polypeptide, a glycoprotein, a glycolipid, a polysaccharide, a protein and a glycosaminoglycan;
X is —O—, —S—, —NH—, or —NR′—;
R′ is hydrogen or C₁₋₅ alkyl;
R is a substituted or unsubstituted C₂ or C₃ alkylene group; and
G is a residue of a second macromolecule selected from the group consisting of an oligonucleotide, a nucleic acid or a metabolically stabilized analogue thereof, a polypeptide, a glycoprotein, a glycolipid, a polysaccharide, a protein and a glycosaminoglycan.

The term “fragment” as used herein refers to the entire molecule itself or a portion or segment of a larger molecule. For example, Y in formula VI may be high molecular weight polysaccharide that is crosslinked by disulfide linkage with another polysaccharide, synthetic polymer, or thiolated polymer to produce the coupled compound. Alternatively, the coupled compound may have multiple disulfide linkages. The compound has at a minimum one unit depicted in formula VI, which represents at least one disulfide linkage as the result of at least one first thiolated compound having the formula I that reacted with at least one second thiolated compound via oxidation.

The macromolecule (Y) and thiolated compound (G) can be any of the macromolecules described above. In one aspect, Y in formula VI is a polysaccharide or a protein. In one aspect, Y is a synthetic polymer. In another aspect, Y in formula VI is a residue of any of the glycosaminoglycans described above including, but not limited to, chondroitin sulfate, dermatan, heparan, heparin, dermatan sulfate, heparan sulfate, alginic acid, chitosan, pectin, or carboxymethylcellulose. In another aspect, G is a residue of any of the polysaccharides described above, including a glycosaminoglycan such as chondroitin sulfate, dermatan, heparan, heparin, dermatan sulfate, heparan sulfate, alginic acid, chitosan, pectin, carboxymethylcellulose, or hyaluronan.

III. Coupling Compounds Via the Reaction Between a Thiol Compound and a Thiol-Reactive Compound

In another aspect, described herein is a method for coupling two or more compounds by reacting a first thiolated macromolecule having the formula I with at least one compound having at one thiol-reactive electrophilic functional group. In one aspect, the compound has at least two-thiol reactive functional groups.

Any of the macromolecules described above can be used in this aspect. Two or more different macromolecules can be used in this method. For example, a second thiolated macromolecule can be used in combination with the first thiolated macromolecule. In this aspect, the first and second thiolated macromolecule can be the same or different compounds.

In one aspect, the first macromolecule is a polysaccharide. In this aspect, the polysaccharide can be a sulfated or non-sulfated glycosaminoglycan including, but not limited to, chondroitin sulfate, dermatan, heparan, heparin, dermatan sulfate, heparan sulfate, alginic acid, chitosan, pectin, or carboxymethylcellulose. In another aspect, the polysaccharide is hyaluronan. In another aspect, when a second macromolecule different from the first macromolecule is used, the second macromolecule can be any of the macromolecules described above having at least one thiol group.

A compound having at least one thiol-reactive electrophilic group is also used in this aspect of the method. The term “thiol-reactive electrophilic group” as used herein is any group that is susceptible to nucleophilic attack by the lone-pair electrons on the sulfur atom of the thiol group or by the thiolate anion of compounds having the formula I as well as other macromolecules. Examples of thiol-reactive electrophilic groups include groups that have good leaving groups. For example, an alkyl group having a halide or alkoxy group attached to it or an α-halocarbonyl group are examples of thiol-reactive electrophilic groups. In another aspect, the thiol-reactive electrophilic group is an electron-deficient vinyl group. The term “an electron-deficient vinyl group” as used herein is a group having a carbon-carbon double bond and an electron-withdrawing group attached to one of the carbon atoms. An electron-deficient vinyl group is depicted in the formula C_{β}═C_{α}X, where X is the electron-withdrawing group. When the electron-withdrawing group is attached to Cα, the other carbon atom of the vinyl group (Cβ) is more susceptible to nucleophilic attack by the thiol group. This type of addition to an activated carbon-carbon double bond is referred to as a Michael addition. Examples of electron-withdrawing groups include, but are not limited to, a nitro group, a cyano group, an ester group, an aldehyde group, a keto group, a sulfone group, or an amide group. Examples of compounds possessing thiol-reactive electrophilic groups include, but are not limited to, maleimides, vinyl sulfones, acrylonitriles, α-methylene esters, quinone methides, acryloyl esters or amides, or α-halo esters or amides.

In one aspect, the thiol-reactive compound has two electron-deficient vinyl groups, wherein the two electron-deficient vinyl groups are the same. In another aspect, the thiol-reactive compound is a diacrylate, a dimethacrylate, a diacrylamide, a dimethacrylamide, or a combination thereof.

In another aspect, the thiol-reactive compound has the formula V 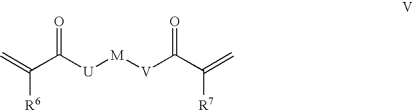
wherein
R⁶ and R⁷ are, independently, hydrogen or lower alkyl;
U and V are, independently, O or NR⁸, wherein R⁸ is, independently, hydrogen or lower alkyl; and
M is a polyalkylene group, a polyether group, a polyamide group, a polyimino group, a polyester, an aryl group, or a polythioether group.

In one aspect, R⁶ and R⁷ are hydrogen, U and V are oxygen, and M is a polyether group. In another aspect, R⁶ and R⁷ are hydrogen, U and V are NH, and M is a polyether group. In a further aspect, R⁶ and R⁷ are methyl, U and V are oxygen, and M is a polyether group. In another aspect, R⁶ and R⁷ are methyl, U and V are NH, and M is a polyether group.

In another aspect, the thiol-reactive compound is any of pharmaceutically-acceptable compounds described above containing at least one thiol-reactive electrophilic group. For example, mitomycin C (MMC) can be converted to the corresponding acrylate (MMC-acrylate). MMC-acrylate is then coupled with a compound having the formula I.

In another aspect, the first thiolated macromolecule has the formula Y—X—R—SH, wherein Y is a residue of a hyaluronan, X is oxygen, and R is —CH₂CH₂— and the thiol-reactive compound has the formula V described above, wherein R⁶ and R⁷ are, independently, hydrogen or lower alkyl; U and V are, independently, O or NR⁸, wherein R⁸ is, independently, hydrogen or lower alkyl; and M is a polyether group.

In one aspect, described herein is a compound having at least one fragment comprising the formula VII 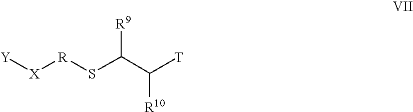
wherein
R⁹ and R¹⁰ are, independently, hydrogen or lower alkyl;
T is an electron-withdrawing group;
Y is a residue of a macromolecule;
X is a residue of a nucleophilic group; and
R comprises a substituted or unsubstituted C₂ or C₃ alkyl group.

In another aspect, described herein is a compound comprising at least one fragment comprises the formula IV 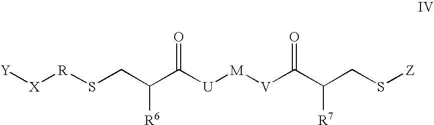
wherein
R⁶ and R⁷ are, independently, hydrogen or lower alkyl;
U and V are, independently, O or NR⁸, wherein R⁸ is, independently, hydrogen or lower alkyl;
Y is a polysaccharide residue or a residue of synthetic polymer;
Z is a residue of a protein;
M is a polyalkylene group, a polyether group, a polyamide group, a polyester group, a polyimino group, an aryl group, or a polythioether group;
X is a residue of a nucleophilic group; and
R comprises a substituted or unsubstituted C₂ or C₃ alkyl group.

In another aspect, with respect to formula IV, Y is a residue of a hyaluronan, X is oxygen, and R is —CH₂CH₂—.

In another aspect, the compound having at least one thiol-reactive group has the formula XX 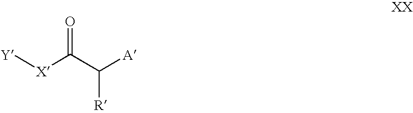
wherein
Y′ is a residue of a macromolecule;
X′ is —O—, —S—, NH— or —NR″—;
R′ is hydrogen, an alkyl group, a perfluoroalkyl group, an aryl group, a heteroaryl group, or a halogen;
R″ is hydrogen or C₁₋₅ alkyl; and
A′ is a leaving group.

The macromolecule residue Y′ in formula XX can be any of the macromolecules described herein. In one aspect, the macromolecule is an oligonucleotide, a nucleic acid or a metabolically stabilized analogue thereof, a polypeptide, a glycoprotein, or a glycolipid. In another aspect, the macromolecule is a polysaccharide, a protein, or a synthetic polymer. With respect to X′, any nucleophilic group present on the macromolecule described herein can be the residue X′.

R′ in formula XX comprises hydrogen, an alkyl group, a perfluoroalkyl group, an aryl group, a heteroaryl group, or a halogen. In one aspect, R′ is hydrogen. In another aspect, R′ is a methyl group.

A′ in formula XX comprises a leaving group. A leaving group is any group that can be displaced by a nucleophile. Several leaving groups are known in the art. Examples include, but are not limited to, halogens, alkoxides, activated esters, and the like. In one aspect, A′ in formula I is chloride, bromide, or iodide.

In one aspect, Y′ comprises a residue of a N-acetyl-glucosamine, wherein at least one primary C-6 hydroxyl group of the N-acetyl-glucosamine residue is substituted with the group —C(O)CH(R)(A′). In another aspect, Y′ comprises a residue of a N-acetyl-glucosamine, wherein at least one primary C-6 hydroxyl group of the N-acetyl-glucosamine residue is substituted with the group —C(O)CH(R)(A′), and at least one secondary hydroxyl group is substituted with the group —C(O)CH(R′)(A′). In a further aspect, Y′ comprises a residue of a N-acetyl-glucosamine, wherein at least one primary C-6 hydroxyl group of the N-acetyl-glucosamine residue is substituted with the group —C(O)CH(R′)(A′), and wherein from one primary C-6 hydroxyl group of the N-acetyl-glucosamine residue to 100% of the primary C-6 hydroxyl groups of the N-acetyl-glucosamine residue are substituted with the group —C(O)CH(R′)(A′). In another aspect, Y′ is a residue of a hyaluronan, wherein at least one hydroxyl group is substituted with —C(O)CH₂Cl, —C(O)CH₂Br, or —C(O)CH₂I.

In one aspect, compounds having the formula XX can be produced by the method comprising reacting a macromolecule comprising at least one nucleophilic group with a compound comprising the formula XV 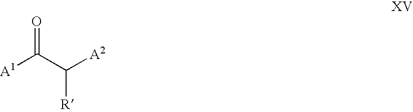
wherein
R′ comprises hydrogen or an alkyl group; and
A¹ and A² comprise the same or different leaving group.

The compounds having the formula XV cover a number of different molecules that can react with a macromolecule. Examples include, but are not limited to, activated esters, acyl halides, anhydrides, and the like.

In one aspect, R′ in formula XV is hydrogen. In another aspect, A¹ in formula XV comprises the formula XVI 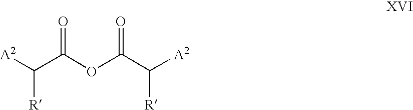
wherein R′ comprises hydrogen or an alkyl group, wherein R′ is the same group; and A² comprises the same leaving group.

Formula XVI covers symmetrical anhydrides; however, as discussed above, mixed anhydrides (e.g., where R′ and/or A² are not the same) are contemplated. In one aspect, R′ in formula XVI is hydrogen. In another aspect, A² in formula XVI comprises a halogen (e.g., chloride, bromide, or iodide). In a further aspect, the compound comprising formula XV is chloroacetic anhydride, bromoacetic anhydride, or iodoacetic anhydride.

Any macromolecule described herein having at least one nucleophilic group including compounds having the formula I can be reacted with the compound having the formula XV to produce a thiol-reactive macromolecule. In certain aspects, the nucleophilic group present on the macromolecule is a hydroxyl group or a substituted or unsubstituted amino group. In one aspect, the macromolecule comprises a glycosaminoglycan such as, for example, hyaluronan. In another aspect, the macromolecule is hyaluronan and the compound having the formula XV is chloroacetic anhydride, bromoacetic anhydride, or iodoacetic anhydride.

The reaction between the macromolecule and the compound having the formula XV can be conducted at various reaction temperatures and times, which will vary depending upon the selection of starting materials. The selection of solvents will also vary on the solubility of the starting materials. In certain aspects, it is desirable to conduct the reaction at a pH greater than 7. For example, when the macromolecule has one or more hydroxyl groups, a basic medium may be desired to deprotonate a certain number of the hydroxyl groups and facilitate the reaction between the macromolecule and the compound having the formula XV.

In one aspect, the reaction between the thiol reactive compound and thiol compound is generally conducted at a pH of from 7 to 12, 7.5 to 11, 7.5 to 10, or 7.5 to 9.5, or a pH of 8. In one aspect, the solvent used can be water (alone) or an aqueous containing organic solvent. In one aspect, when the mixed solvent system is used, a base such as a primary, secondary, or tertiary amine can be used. In one aspect, an excess of thiol compound is used relative to the thiol-reactive compound in order to ensure that all of the thiol-reactive compound is consumed during the reaction. Depending upon the selection of the thiol reactive compound, the thiol compound, the pH of the reaction, and the solvent selected, coupling can occur from within minutes to several days. If the reaction is performed in the presence of an oxidant, such as air, the thiol compound can react with itself or another thiol compound via oxidative addition to form a disulfide linkage in addition to reacting with the thiol-reactive compound.

IV. Pharmaceutical Compositions

In one aspect, any of the compounds produced by the methods described above (e.g., compounds having the formula I and crosslinked compounds) can be used a pharmaceutical. In another aspect, any of the compounds produced by the methods described above (e.g., compounds having the formula I and crosslinked compounds) can include or be combined with at least one pharmaceutically-acceptable compound. The resulting pharmaceutical composition can provide a system for sustained, continuous delivery of drugs and other biologically-active agents to tissues adjacent to or distant from the application site. The biologically-active agent is capable of providing a local or systemic biological, physiological or therapeutic effect in the biological system to which it is applied. For example, the agent can act to control infection or inflammation, enhance cell growth and tissue regeneration, control tumor growth, act as an analgesic, promote anti-cell attachment, and enhance bone growth, among other functions. Additionally, any of the compounds described herein can contain combinations of two or more pharmaceutically-acceptable compounds.

In one aspect, the pharmaceutically-acceptable compounds can include substances capable of preventing an infection systemically in the biological system or locally at the defect site, as for example, anti-inflammatory agents such as, but not limited to, pilocarpine, hydrocortisone, prednisolone, cortisone, diclofenac sodium, indomethacin, 6∝-methyl-prednisolone, corticosterone, dexamethasone, prednisone, and the like; antibacterial agents including, but not limited to, penicillin, cephalosporins, bacitracin, tetracycline, doxycycline, gentamycin, chloroquine, vidarabine, and the like; analgesic agents including, but not limited to, salicylic acid, acetaminophen, ibuprofen, naproxen, piroxicam, flurbiprofen, morphine, and the like; local anesthetics including, but not limited to, cocaine, lidocaine, benzocaine, and the like; immunogens (vaccines) for stimulating antibodies against hepatitis, influenza, measles, rubella, tetanus, polio, rabies, and the like; peptides including, but not limited to, leuprolide acetate (an LH-RH agonist), nafarelin, and the like. All compounds are commercially available.

Additionally, a substance or metabolic precursor which is capable of promoting growth and survival of cells and tissues or augmenting the functioning of cells is useful, as for example, a nerve growth promoting substance such as a ganglioside, a nerve growth factor, and the like; a hard or soft tissue growth promoting agent such as fibronectin (FN), human growth hormone (HGH), a colony stimulating factor, bone morphogenic protein, platelet-derived growth factor (PDGF), insulin-derived growth factor (IGF-I, IGF-II), transforming growth factor-alpha (TGF-alpha), transforming growth factor-beta (TGF-beta), epidermal growth factor (EGF), fibroblast growth factor (FGF), interleukin-1 (IL-1), vascular endothelial growth factor (VEGF) and keratinocyte growth factor (KGF), dried bone material, and the like; and antineoplastic agents such as methotrexate, 5-fluorouracil, adriamycin, vinblastine, cisplatin, tumor-specific antibodies conjugated to toxins, tumor necrosis factor, and the like.

Other useful substances include hormones such as progesterone, testosterone, and follicle stimulating hormone (FSH) (birth control, fertility-enhancement), insulin, and the like; antihistamines such as diphenhydramine, and the like; cardiovascular agents such as papaverine, streptokinase and the like; anti-ulcer agents such as isopropamide iodide, and the like; bronchodilators such as metaproternal sulfate, aminophylline, and the like; vasodilators such as theophylline, niacin, minoxidil, and the like; central nervous system agents such as tranquilizer, B-adrenergic blocking agent, dopamine, and the like; antipsychotic agents such as risperidone, narcotic antagonists such as naltrexone, naloxone, buprenorphine; and other like substances. All compounds are commercially available.

The pharmaceutical compositions can be prepared using techniques known in the art. In one aspect, the composition is prepared by admixing a compound described herein with a pharmaceutically-acceptable compound. The term “admixing” is defined as mixing the two components together so that there is no chemical reaction or physical interaction. The term “admixing” also includes the chemical reaction or physical interaction between the compound and the pharmaceutically-acceptable compound.

It will be appreciated that the actual preferred amounts of active compound in a specified case will vary according to the specific compound being utilized, the particular compositions formulated, the mode of application, and the particular situs and subject being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

Pharmaceutical compositions described herein can be formulated in any excipient the biological system or entity can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity-enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol. In one aspect, the compounds described herein are admixed with a non-FDA approved delivery device such as, for example, sunscreen or a nutraceutical.

Pharmaceutical carriers are known to those skilled in the art. These most typically would be standard carriers for administration to humans, including solutions such as sterile water, saline, and buffered solutions at physiological pH.

Molecules intended for pharmaceutical delivery can be formulated in a pharmaceutical composition. Pharmaceutical compositions can include carriers, thickeners, diluents, buffers, preservatives, surface-active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally).

Preparations for administration include sterile aqueous or non-aqueous solutions, suspensions, and emulsions. Examples of non-aqueous carriers include water, alcoholic/aqueous solutions, emulsions or suspensions, including saline and buffered media. Parenteral vehicles, if needed for collateral use of the disclosed compositions and methods, include sodium chloride solution, Ringers dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Dosing is dependent on severity and responsiveness of the condition to be treated, but will normally be one or more doses per day, with course of treatment lasting from several days to several months or until one of ordinary skill in the art determines the delivery should cease. Persons of ordinary skill can easily determine optimum dosages, dosing methodologies and repetition rates.

In one aspect, any of the compounds and pharmaceutical compositions can include living cells. Examples of living cells include, but are not limited to, fibroblasts, hepatocytes, chondrocytes, stem cells, bone marrow, muscle cells, cardiac myocytes, neuronal cells, or pancreatic islet cells.

Depending upon the selection of the compound having the formula I, the compound may not form a hydrogel when added to water. For example when Y is a residue of hyaluronan, X is oxygen, and R is —CH₂CH₂—, little to no hydrogel formation occurs when it is added to water. This can be desirable in certain applications, particularly when the compound is administered by injection or intravenously.

V. Methods of Use

The compounds and pharmaceutical compositions described herein (e.g., compounds having the formula I and crosslinked compounds derived from compounds having the formula I) have a variety of uses related to drug delivery, small molecule delivery, wound healing, burn injury healing, anti-inflammation, and cell/tissue engineering. In certain aspects, the disclosed compositions are useful for situations that benefit from a hydrated, pericellular environment in which assembly of other matrix components, presentation of growth and differentiation factors, cell migration, or tissue regeneration are desirable.

In one aspect, described herein are methods for reducing or preventing inflammation in a subject with inflammation or at risk for inflammation, comprising administering an effective amount of one or more compounds described herein, the compound reducing or preventing the inflammation in the subject. The methods described herein contemplate the use of single or mixtures of two or more compounds described herein. The compounds can be administered using the techniques described above. Examples of inflammation include, but are not limited to, pulmonary inflammation, vascular inflammation, renal inflammation, inflammation of the central nervous system, hepatic inflammation, inflammation in a joint, or splanchnic inflammation. The inflammation can be associated with an inflammatory disease including, but not limited to, systemic lupus erythematosus, Hashimoto's disease, rheumatoid arthritis, graft-versus-host disease, Sjogren's syndrome, pernicious anemia, Addison disease, scleroderma, Goodpasture's syndrome, Crohn's disease, autoimmune hemolytic anemia, myasthenia gravis, multiple sclerosis, Alzheimer's disease, amyotrophic lateral sclerosis, Basedow's disease, thrombopenia purpura, insulin-dependent diabetes mellitus, allergy; asthma, inflammatory bowel disease, cancer, ulcerative colitis, scleroderma, cardiomyopathy, atherosclerosis, hypertension, sickle cell disease, or respiratory distress syndrome of neonate and adults. In another aspect, the inflammation can be caused by an organ transplantation, respiratory distress, ventilator induced lung injury, ischemia reperfusion, hemorrhagic shock, or sepsis. In one aspect, when the pulmonary inflammation is caused by respiratory distress or sepsis, the nitrated lipids can reduce or prevent the accumulation of alveolar fluid in a subject. In another aspect, described herein is a method for preventing or reducing ischemic reperfusion in a tissue (e.g., liver, kidney, cardiovascular) of a subject, comprising contacting the tissue with a compound having the formula I.

As described above, free radical and reactive oxygen species present in a subject can cause inflammation, pain, and cell/tissue damage or death. In one aspect, the compounds having the formula I can reduce or prevent damage to a cell or tissue caused by a free radical or reactive oxygen species, wherein the method comprises contacting the cell with of one or more compounds having the formula I. The free radicals or reactive oxygen species can be endogenous or produced by external means. Examples of reactive oxygen species include, but are not limited to, NO., HO., HOO.⁻, HOO., or O₂.⁻. Free radical or reactive oxygen species can be produced by exposure of the cell to radiation. Such exposure can involve radiation in medical procedures with respect to tumor reduction, radiation exposure from the sun, radiation exposure in the military, civilian exposure to radiation (e.g., at power plants), and the like. In one aspect, described herein is a method for protecting skin from exposure to a reactive oxygen species, comprising contacting the skin with a compound having the formula I.

The compounds described herein can be used to preserve and protect organs, tissue, and cells from damage caused by free radical and reactive oxygen species. In one aspect, described herein are methods for reducing or preventing the formation of scar tissue in a subject produced by a free radical or reactive oxygen species, comprising administering an effective amount of one or more compounds having the formula I, the compound reducing or preventing the formation of scar tissue in the subject. The method can be performed in vivo or ex vivo. In ex vivo applications, the compounds having the formula I can be used to preserve an organ or tissue that is susceptible to damage caused by free radical or reactive oxygen species. In one aspect, the compounds described herein can preserve and/or protect adult/embryonic stem cells, sperm cells, and the like.

The compounds and compositions described herein can deliver at least one pharmaceutically-acceptable compound to a patient in need of such delivery, comprising contacting at least one tissue capable of receiving the pharmaceutically-acceptable compound with one or more compositions described herein. The compounds described herein can be used as a carrier for a wide variety of releasable biologically active substances having curative or therapeutic value for human or non-human animals. Many of these substances that can be carried by the compound are discussed above. Included among biologically active materials which are suitable for incorporation into the gels of the invention are therapeutic drugs, e.g., anti-inflammatory agents, anti-pyretic agents, steroidal and non-steroidal drugs for anti-inflammatory use, hormones, growth factors, contraceptive agents, antivirals, antibacterials, antifungals, analgesics, hypnotics, sedatives, tranquilizers, anti-convulsants, muscle relaxants, local anesthetics, antispasmodics, antiulcer drugs, peptidic agonists, sympathiomimetic agents, cardiovascular agents, antitumor agents, oligonucleotides and their analogues and so forth. A biologically active substance is added in pharmaceutically active amounts.

In one aspect, the compounds and compositions described herein can be used for the delivery of living cells to a subject. Any of the living cells described above can be used in the aspect.

In one aspect, the compounds and compositions can be used for the delivery of growth factors and molecules related to growth factors. For example the growth factors can be a nerve growth promoting substance such as a ganglioside, a nerve growth factor, and the like; a hard or soft tissue growth promoting agent such as fibronectin (FN), human growth hormone (HGH), a colony stimulating factor, bone morphogenic protein, platelet-derived growth factor (PDGF), insulin-derived growth factor (IGF-I, IGF-II), transforming growth factor-alpha (TGF-alpha), transforming growth factor-beta (TGF-beta), epidermal growth factor (EGF), fibroblast growth factor (FGF), interleukin-1 (IL-1). Preferred growth factors are bFGF and TGF-β.

Also preferred are vascular endothelial growth factor (VEGF) and keratinocyte growth factor (KGF).

Described herein are methods for improving wound healing in a subject in need of such improvement by contacting any of the compounds or pharmaceutical compositions described herein with a wound of a subject in need of wound healing improvement. Also provided are methods to deliver at least one pharmaceutically-acceptable compound to a patient in need of such delivery by contacting any of the compounds or pharmaceutical compositions described herein with at least one tissue capable of receiving said pharmaceutically-acceptable compound.

The compounds and pharmaceutical compositions described herein can be placed directly in or on any biological system without purification as it is composed of biocompatible materials. Examples of sites the compounds can be placed include, but not limited to, soft tissue such as muscle or fat; hard tissue such as bone or cartilage; areas of tissue regeneration; a void space such as periodontal pocket; surgical incision or other formed pocket or cavity; a natural cavity such as the oral, vaginal, rectal or nasal cavities, the cul-de-sac of the eye, and the like; the peritoneal cavity and organs contained within, and other sites into or onto which the compounds can be placed including a skin surface defect such as a cut, scrape or burn area. It is contemplated that the tissue can be damaged due to injury or a degenerative condition or, in the alternative, the compounds and compositions described herein can be applied to undamaged tissue to prevent injury to the tissue. The present compounds can be biodegradable and naturally occurring enzymes will act to degrade them over time. Components of the compound can be “bioabsorbable” in that the components of the compound will be broken down and absorbed within the biological system, for example, by a cell, tissue and the like. Additionally, the compounds, especially compounds that have not been rehydrated, can be applied to a biological system to absorb fluid from an area of interest.

The disclosed compositions can be used for treating a wide variety of tissue defects in an animal, for example, a tissue with a void such as a periodontal pocket, a shallow or deep cutaneous wound, a surgical incision, a bone or cartilage defect, and the like. For example, the cross-linked compounds described herein can be in the form of a hydrogel film. The hydrogel film can be applied to a defect in bone tissue such as a fracture in an arm or leg bone, a defect in a tooth, a cartilage defect in the joint, ear, nose, or throat, and the like. The hydrogel film composed of the compound described herein can also function as a barrier system for guided tissue regeneration by providing a surface on or through which the cells can grow. To enhance regeneration of a hard tissue such as bone tissue, it is preferred that the hydrogel film provides support for new cell growth that will replace the matrix as it becomes gradually absorbed or eroded by body fluids.

The use of the compounds describe above to prevent adhesion after a surgical procedure, wherein the surgical procedure comprises cardiosurgery and articular surgery, abdominal surgery, a surgical procedure performed in the urogenital region, a surgical procedure involving a tendon, ligament, rotator cuff, laparascopic surgery, pelvic surgery, oncological surgery, sinus and craniofacial surgery, ENT surgery, a procedure involving spinal dura repair, or for vocal fold repair, prophylaxis, or restoration of function.

The hydrogel film composed of a compound described herein can be delivered onto cells, tissues, and/or organs, for example, by injection, spraying, squirting, brushing, painting, coating, and the like. Delivery can also be via a cannula, catheter, syringe with or without a needle, pressure applicator, pump, and the like. The compound can be applied onto a tissue in the form of a film, for example, to provide a film dressing on the surface of the tissue, and/or to adhere to a tissue to another tissue or hydrogel film, among other applications.

In one aspect, the compounds described herein are administered via injection. For many clinical uses, when the compound is in the form of a hydrogel film, injectable hydrogels are preferred for three main reasons. First, an injectable hydrogel could be formed into any desired shape at the site of injury. Because the initial hydrogels can be sols or moldable putties, the systems can be positioned in complex shapes and then subsequently crosslinked to conform to the required dimensions. Second, the hydrogel would adhere to the tissue during gel formation, and the resulting mechanical interlocking arising from surface microroughness would strengthen the tissue-hydrogel interface. Third, introduction of an in situ-crosslinkable hydrogel could be accomplished using needle or by laparoscopic methods, thereby minimizing the invasiveness of the surgical technique.

The compounds described herein can be used to treat periodontal disease, gingival tissue overlying the root of the tooth can be excised to form an envelope or pocket, and the composition delivered into the pocket and against the exposed root. The compounds can also be delivered to a tooth defect by making an incision through the gingival tissue to expose the root, and then applying the material through the incision onto the root surface by placing, brushing, squirting, or other means.

When used to treat a defect on skin or other tissue, the compounds described herein can be in the form of a hydrogel film that can be placed on top of the desired area. In this aspect, the hydrogel film is malleable and can be manipulated to conform to the contours of the tissue defect.

It is understood that the disclosed compositions and methods can be applied to a subject in need of tissue regeneration. For example, cells can be incorporated into the compounds described herein for implantation. In one aspect the subject is a mammal. Preferred mammals to which the compositions and methods apply are mice, rats, cows or cattle, horses, sheep, goats, cats, dogs, ferrets, and primates, including apes, chimpanzees, orangatangs, and humans. In another aspect, the compounds and compositions described herein can be applied to birds.

When being used in areas related to tissue regeneration such as wound or burn healing, it is not necessary that the disclosed methods and compositions eliminate the need for one or more related accepted therapies. It is understood that any decrease in the length of time for recovery or increase in the quality of the recovery obtained by the recipient of the disclosed compositions or methods has obtained some benefit. It is also understood that some of the disclosed compositions and methods can be used to prevent or reduce fibrotic adhesions occurring as a result of wound closure as a result of trauma, such surgery. It is also understood that collateral affects provided by the disclosed compositions and compounds are desirable but not required, such as improved bacterial resistance or reduced pain etc.

The compounds described herein can be used as substrates for growing and differentiating cells. For example, the compounds and compositions described herein can be formed into a laminate, a gel, a bead, a sponge, a film, a mesh, an electrospun nanofiber, or a matrix.

In one aspect, described herein is a method for growing a plurality of cells, comprising (a) depositing a parent set of cells on a substrate described herein, and (b) culturing the substrate with the deposited cells to promote the growth of the cells. In another aspect, described herein is a method for differentiating cells, comprising (a) depositing a parent set of cells on a substrate described herein, and (b) culturing the assembly to promote differentiation of the cells.

Many types of cells can be grown and/or differentiated using the substrates described herein including, but not limited to, stem cells, committed stem cells, differentiated cells, and tumor cells. Examples of stem cells include, but are not limited to, embryonic stem cells, bone marrow stem cells and umbilical cord stem cells. Other examples of cells used in various embodiments include, but are not limited to, osteoblasts, myoblasts, neuroblasts, fibroblasts, glioblasts, germ cells, hepatocytes, chondrocytes, epithelial cells, cardiovascular cells, keratinocytes, smooth muscle cells, cardiac muscle cells, connective tissue cells, glial cells, epithelial cells, endothelial cells, hormone-secreting cells, cells of the immune system, and neurons.

Cells useful herein can be cultured in vitro, derived from a natural source, genetically engineered, or produced by any other means. Any natural source of prokaryotic or eukaryotic cells can be used. It is also contemplated that cells can be cultured ex vivo.

Atypical or abnormal cells such as tumor cells can also be used herein. Tumor cells cultured on substrates described herein can provide more accurate representations of the native tumor environment in the body for the assessment of drug treatments. Growth of tumor cells on the substrates described herein can facilitate characterization of biochemical pathways and activities of the tumor, including gene expression, receptor expression, and polypeptide production, in an in vivo-like environment allowing for the development of drugs that specifically target the tumor.

Cells that have been genetically engineered can also be used herein. The engineering involves programming the cell to express one or more genes, repressing the expression of one or more genes, or both. Genetic engineering can involve, for example, adding or removing genetic material to or from a cell, altering existing genetic material, or both. Embodiments in which cells are transfected or otherwise engineered to express a gene can use transiently or permanently transfected genes, or both. Gene sequences may be full or partial length, cloned or naturally occurring.

In another aspect, described herein is method for growing tissue, comprising (a) depositing a parent set of cells that are a precursor to the tissue on a substrate described herein, and (b) culturing the substrate with the deposited cells to promote the growth of the tissue. It is also contemplated that viable cells can be deposited on the substrates described herein and cultured under conditions that promote tissue growth. Tissue grown (i.e., engineered) from any of the cells described above is contemplated with the substrates described herein. The supports described herein can support many different kinds of precursor cells, and the substrates can guide the development of new tissue. The production of tissues has numerous applications in wound healing. Tissue growth can be performed in vivo or ex vivo using the methods described herein.

The compounds described herein can be applied to an implantable device such as a suture, clamps, prosthesis, catheter, stents, metal screw, bone plate, pin, a bandage such as gauze, and the like, to enhance the compatibility and/or performance or function of an implantable device with a body tissue in an implant site. The compounds can be used to coat the implantable device. For example, the compounds could be used to coat the rough surface of an implantable device to enhance the compatibility of the device by providing a biocompatible smooth surface that reduces the occurrence of abrasions from the contact of rough edges with the adjacent tissue. The compounds can also be used to enhance the performance or function of an implantable device. For example, when the compound is a hydrogel film, the hydrogel film can be applied to a gauze bandage to enhance its compatibility or adhesion with the tissue to which it is applied. The hydrogel film can also be applied around a device such as a catheter or colostomy that is inserted through an incision into the body to help secure the catheter/colostomy in place and/or to fill the void between the device and tissue and form a tight seal to reduce bacterial infection and loss of body fluid. In one aspect, the compounds can be coated onto metal stents (titanium, nickel, gold, etc.) used in angioplasty (atherosclerosis) and prevent restenosis by preventing scar tissue formation. In another aspect, the compounds described herein can be used to coat metal joints.

It is understood that any given particular aspect of the disclosed compositions and methods can be easily compared to the specific examples and embodiments disclosed herein, including the non-polysaccharide based reagents discussed in the Examples. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Materials and Methods

Materials and analytical instrumentation. High molecular weight hyaluronan (HA, MW=824 kDa) was from Novozymes Biopolymers. Ethylene sulfide and 5,5′-dithiobis(2-nitrobenzoic acid) were from Aldrich Chemical Co. (Milwaukee, Wis.). 10× Phosphate buffered saline (PBS), sodium hydroxide (NaOH), hydrochloric acid 12.1 N(HCl), sodium iodide (NaI), dibasic sodium phosphate, heptahydrate (Na₂PO₄.7H₂O) and SpectraPor dialysis tubing MWCO 10,000 were from Fisher Scientific (Hanover Park, Ill.). 5-((2-(and-3)-S-acetylmercapto)succinoyl)amino) fluorescein (SAMSA fluorescein) mixed isomers was purchased from Molecular Probes Inc. (Eugene, Oreg.). Dithiothreitol (DTT) was from BioVectra DCL (Charlottetown, PE, Canada). ¹H-NMR spectral data was acquired using a Varian INOVA 400 at 400 MHz. UV/VIS spectra and measurements were performed on a Hewlett-Packard 8453 UV-visible spectrometer (Palo Alto, Calif.)

Synthesis of 2-Thioethyl Ether Derived Hyaluronan (HASH) (Procedure 1). 400 mg hyaluronan (824 kDa) was dissolved in 40 ml distilled water (1% w/v solution). The pH of the solution was raised to 9.16 by adding 5M NaOH. A 5 fold molar excess of ethylene sulfide was added to the HA solution and the reaction mixture was stirred overnight at room temperature. Some precipitation was observed due to ethylene sulfide polymerization. A small amount of activated carbon was added and the reaction volume was increased by adding 200 ml distilled water to decrease viscosity. The solution was then filtered. 5 fold molar excess of DTT was added to the clear filtrate and the pH of the solution was raised to 8.55 with 5M NaOH. The reaction was stirred overnight at room temperature. After 24 hours, the pH of the reaction mixture was decreased to 3.5 by adding 6N HCl. The acidified solution was dialyzed (MWCO 10000) against dilute HCl (pH 3.5) containing 100 mM NaCl, followed by dialysis against dilute HCl (pH 3.5). Next, the solution was lyophilized, the purity of the sample was determined by ¹H-NMR and the degree of substitution was determined by derivatization with SAMSA fluorescein and ¹H-NMR. Yield=78%; m=1.97 g. Degree of substitution 53% (¹H-NMR). MW=200 kDa (GPC).

Synthesis of 2-Thioethyl Ether Derived Hyaluronan (HA-TEE) (Procedure 2). 2 g hyaluronan (824 kDa) was dissolved in 400 ml distilled water (0.5% w/v solution). The pH of the solution was raised to 10.0 by adding 1M NaOH. A 5-fold molar excess of ethylene sulfide was added dropwise to the HA solution under aggressive stirring and the reaction mixture was allowed to proceed for 24 h at room temperature. Some precipitation was observed due to ethylene sulfide polymerization. The reaction mixture was subsequently vacuum filtered on a one-inch bed of Celite 545 (Sigma). Next, a 5-fold molar excess of DTT was added to the clear filtrate and the pH of the solution was raised to 8.5 with 1M NaOH. The reaction was stirred overnight at room temperature. After 24 hours, the pH of the reaction mixture was decreased to 3.5 by adding 6N HCl. The acidified solution was dialyzed (MWCO 10,000) against dilute HCl (pH 3.5). Next, the solution was lyophilized and the purity of the sample was determined by ¹H-NMR. GPC was used to determine the molecular weight (MW˜170 kDa) and polydispersity index (PI˜1.9) of the new material. Both values were found to be in good agreement with the previously obtained material. Yield=72%; m=1.44 g.

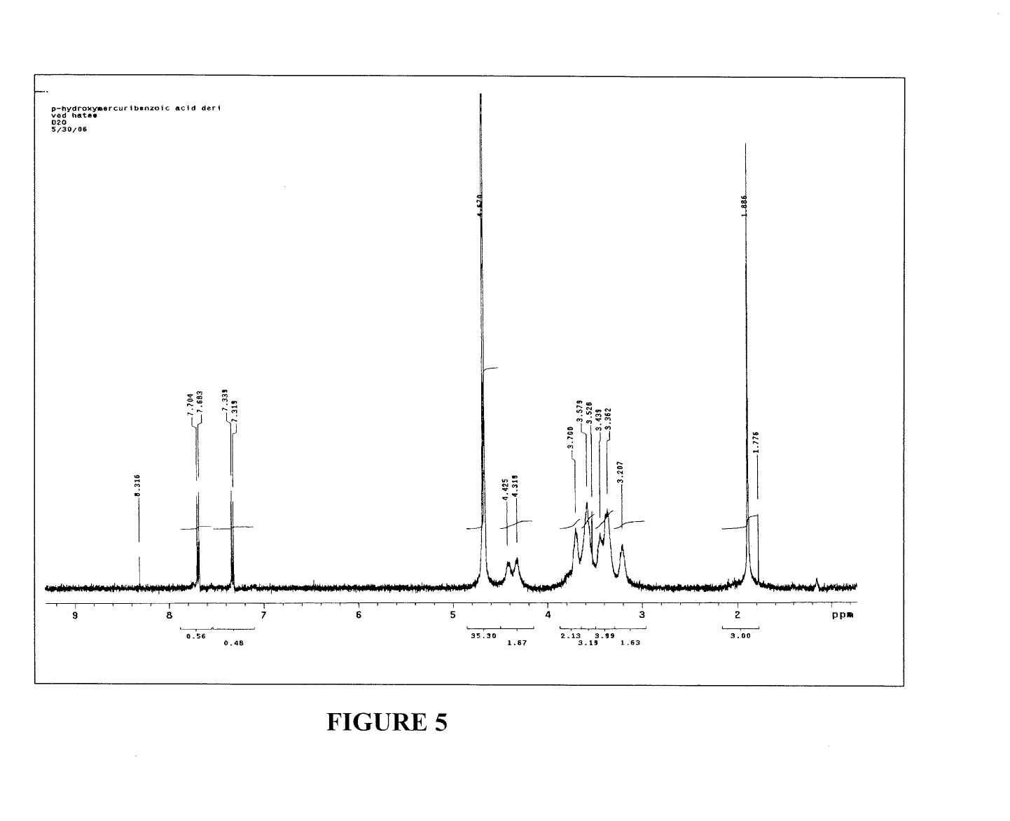

HO—Hg—C₆H₄—COONa (4-(hydroxymercuri)benzoic acid sodium salt) derivatization. 1% (w/v) HASH solution was reacted with 4-(hydroxymercuric)benzoic acid sodium salt for 24 h at room temperature. The reaction stoichiometry was 1:1 (disaccharide unit: reagent). The precipitated reagent was then removed by filtration and the filtrate was analyzed by ¹H-NMR. FIG. 5 shows the ¹H-NMR analysis of 4-(hydroxymercuri)benzoic acid sodium salt derivatized HASH.

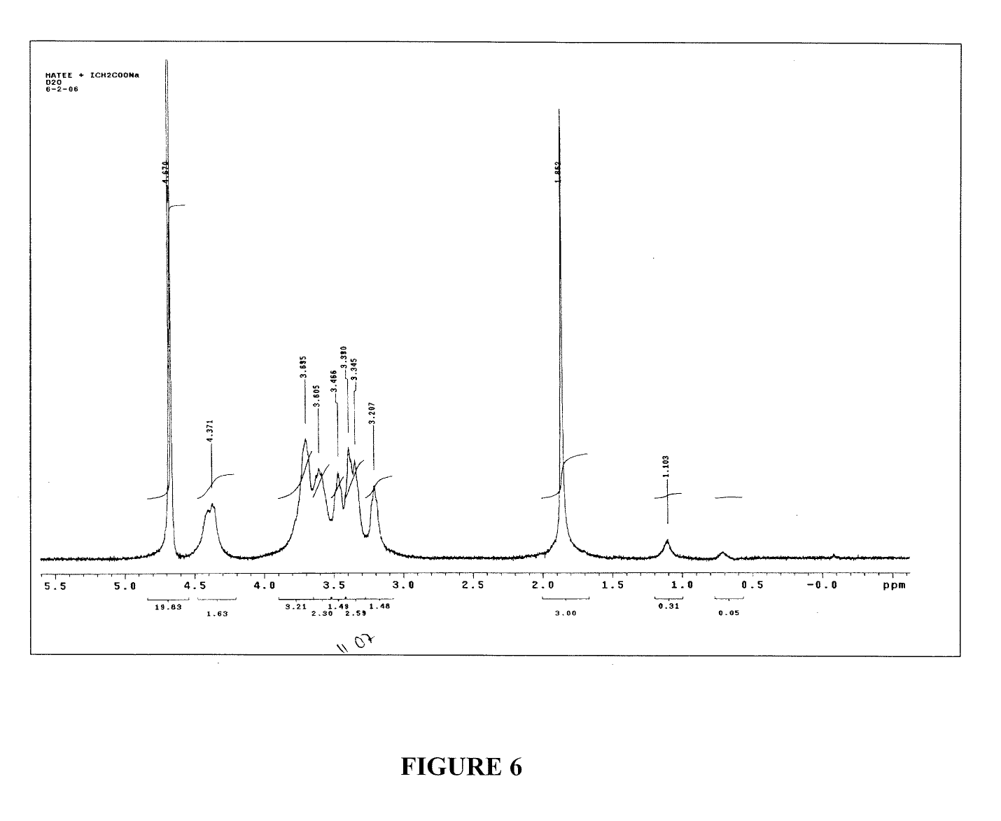

ICH₂COONa (sodium iodoacetate) derivatization. 1% (w/v) HASH solution was reacted with sodium iodoacetate for 24 h at room temperature. The reaction stoichiometry was 1:1 (disaccharide unit to reagent). The reaction mixture was dialyzed 2 days against distilled water. Subsequently, the reaction product was lyophilized. FIG. 6 shows the ¹H-NMR analysis of sodium iodoacetate derivatized HASH.

Thiol Content Determination. HASH (24 mg) was dissolved in 8 mL DTNB solution (2 mg/mL in 0.1 M PBS, pH 8.0) and the solution was stirred overnight at room temperature followed by subsequent dialysis for 3 days (Slide-A-Lyzer 10 K dialysis cassette, Pierce, Rockford, Ill.). The derivatized HASH was then lyophilized and 2 mg of the lyophilized material was then dissolved in 1 mL 0.1 M PBS, pH 7.4. 2.5 mL DTT solution (1% w/w DTT in dH₂O, pH 8.5) was added to 0.1 mL TNB-HASH solution. After the mixture turned yellow, the A₄₁₂ was determined using a Hewlett-Packard 8453 UV-visible spectrometer (Palo Alto, Calif.).

Attempted Crosslinking of HASH. HASH solutions (2% and 2.5% w/v) were prepared in 1×PBS buffer and the pH of the solutions was adjusted to 6, 7, 8, 9 and 10 (the pKa range of thiol groups). Crosslinker solutions (4%, 8% and 10% w/v) were used for crosslinking experiments, and Table 1 summarizes the bivalent electrophiles or oxidants evaluated. As positive control, a thiol-derivatized carboxymethylated HA (CMHA-S) was used (Table 2). HASH and crosslinker solutions were mixed in different molar ratios (1:1, 1:2, 1:3, 2:1, 3:1; 4:1 and 5:1) and set at room temperature. Gelation was monitored by using the test tube inversion assay. No gelation was observed for any of the tested conditions, even after 48 h (Table 3).

TABLE 1
Structures of bivalent thiol-reactive crosslinkers evaluated with thiol-modified
HA derivatives. All PEG derivatives were prepared from PEG 3400.
Crosslinkers tested

| Crosslinker | Structure |
|---|---|
| Polyethylene glycol diacrylate (PEGDA) | 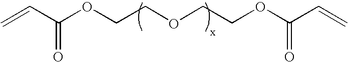 |
| Polyethylene glycol bisbromoacetate (PEGDBrAc) | 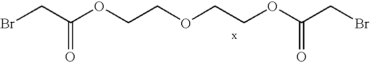 |
| Polyethylene glycol bisiodoacetate (PEGDIAc) | 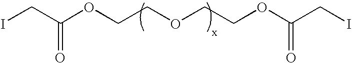 |
| Polyethylene glycol bismaleimide (PEGDMal) | 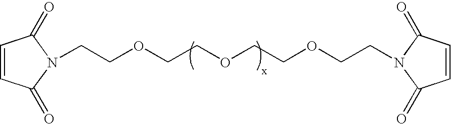 |
| HS-PEG-SH | 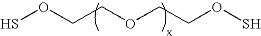 |
| Hyaluronan Bromoacetate (HABA) | 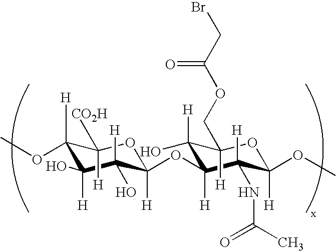 |
| Hyaluronan Iodoacetate (HAIA) | 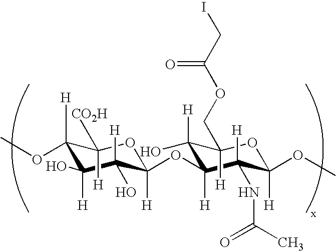 |
| Hydrogen peroxide | H₂O₂ (crosslinking agent) |

SAMSA Fluorescein Derivatization. 4 mg of SAMSA fluorescein was dissolved in 400 μl 0.1 M NaOH and incubate for 15 minutes at room temperature. 5.6 μl 6N HCl were then added followed by the addition of 80 μl NaH₂PO₄.H₂O, pH 7.0. HA and HA-TEE were each reacted with 5 fold excess of activated SAMSA fluorescein for 30 minutes at room temperature. The reaction mixtures were then dialyzed (MWCO 2,000) against dilute NaOH (pH 9.0) for 3 days. The A_{495 nm} and the fluorescence of the SAMSA derivatized compounds were determined together with a 200 nm to 800 nm scan. The degree of chemical modification of the HA polymers was determined by using Lambert-Beer equation (extinction coefficient of SAMSA 80000 M⁻¹ cm⁻¹). SD=10% (the inconsistencies in the degrees of substitution calculated with the two methods—¹H-NMR and SAMSA derivatization—are most probably due to the decreased chemical reactivity of the thiol group caused by the relative shortness of the substituent chain and the steric hindrance of the HA molecule).

HASHHASH Cytotoxicity Assay. Primary human tracheal scar fibroblasts (T31 cells) were seeded in a 96-well plate (seeding density was 12.5×10³ cells/well in 100 μl) in DMEM/F12+10% newborn calf serum+2 mM L-glutamine+penicillin/streptomycin. Cells were allowed to recover and attach for 24 h at 37° C./5% CO₂. The next day, the media was replaced with DMEM/F12 containing 1.5%, 1%, 0.6%, 0.2% and 0.1% HA and HASH, respectively. Cells were incubated for an additional 24 h and cell viability in the presence or absence of HASH was assessed using a previously described biochemical method. The tetrazolium compound MTS (Cell-Titer 96 Aqueous One Solution Cell Proliferation Assay, Promega, Madison, Wis.) is reduced by metabolically active cells to yield a colored formazan product, and the absorption at 490 nm is proportional to the number of viable cells.

Gelation studies. 2.5% HASH solutions were made at pH ε [7-10] (the pH range of thiol groups) in 1×PBS buffer. 10% polyethylene glycol derivative solutions in 1×PBS (PEG diacrylate, bisbromoacetate, PEG bisiodoacetate and PEG bismaleimide) were used for crosslinking experiments. HASH and crosslinker solutions were mixed in different volume ratios (3:1; 4:1:5:1) and set at room temperature. No gelation was observed under any of the tested conditions.

Chondrocyte Culture and Treatment. Articular chondrocytes were obtained from the knee joints of a 2-year old sheep immediately postmortem. The tissue was first minced then treated overnight with 0.1% type II collagenase. The isolated cells were then grown in DMEM/F12+10% FBS+penicillin/streptomycin at 37° C./5% CO₂. The medium was changed at confluence to DMEM/F12+0.5% FBS+penicillin/streptomycin, for 6 h. Subsequently, chondrocytes were treated with HA and HASH, respectively (0, 50, 100 and 200 μg/mL final concentrations). After 2 h, H₂O₂ was added to the medium to a final concentration of 0.5 mM, for 24 h. As controls, chondrocytes were cultured in medium alone or medium plus H₂O₂.

Determination of Apoptosis by Flow Cytometry Analysis. The apoptotic rate of chondrocytes was evaluated with an Annexin V-FITC kit. After apoptosis induction, cells were washed twice with 1× PBS then were suspended in 1× binding buffer at a density of 10⁶ cells/ml. Annexin V-FITC and propidium iodide were used to stain cells, at room temperature for 15 min. Samples were further 5-fold diluted with 1× binding buffer and analyzed by flow cytometry. Cell populations were identified as follows: intact (Annexin V-FITC⁻, propidium iodide⁻), early apoptotic (Annexin V-FITC⁺, propidium iodide⁻), late apoptotic and necrotic (Annexin V-FITC⁺, propidium iodide⁺).

Statistical analysis. The data is represented as the means ± standard deviation (S.D.) of number of repeats. Values were compared using Student's t-test (2-tailed) with p<0.05 considered statistically significant and p<0.005 considered highly significant.

Synthesis and Characterization of 2-Thioethyl Ether Hyaluronan (HASH). Thiolated HA derivatives were previously synthesized in our laboratory via hydrazide chemistry. This strategy targeted the glucuronic acid (GlcA) residues of GAG disaccharide units. The first step in the procedure involved the reaction of the GlcA carboxyl groups with 3,3′-di(thiopropionyl) bishydrazide) (DTP) in the presence of 1-ethyl-3-[3-dimethylamino)propyl]carbodiimide (EDCI). The resulting disulfide-containing GAGs were subsequently reduced with dithiothreitol (DTT) yielding the thiolated macromoleculates.

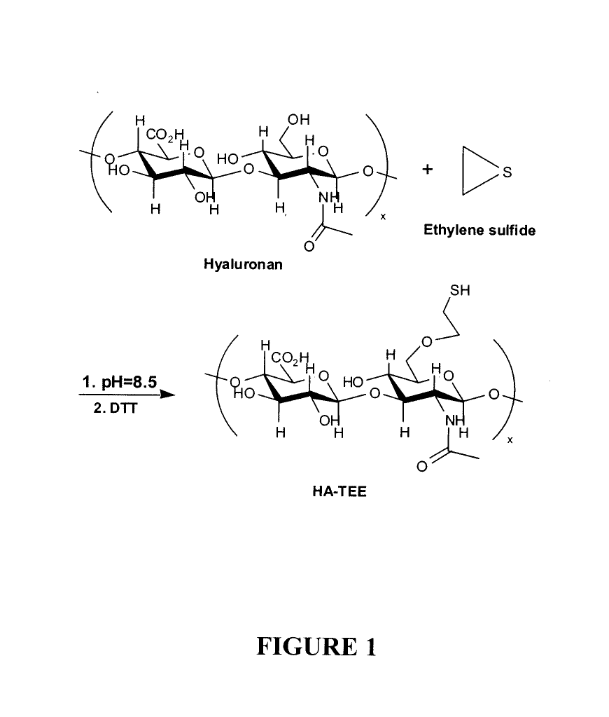

For the synthesis of HASH, the approach was to chemically alter the reactive primary hydroxyl group of the N-acetyl glucosamine (GlcNAc) residues of HA by the nucleophilic opening of ethylene sulfide with alkoxides transiently formed at basic pH (FIG. 1). This strategy is analogous to the base-mediated carboxymethylation of HA, or the partial crosslinking of HA using divinyl sulfone crosslinked HA or the reaction with 1,4-butanediol diglycidyl ether. Subsequently, the reaction mixture was treated with DTT to reduce any residual disulfide bonds, followed by dialysis and lyophilization.

While it is plausible to consider that the carboxylate could open the ethylene sulfide, this reaction is reversible. Any (2-thioethyl) ester formed would rapidly undergo beta-elimination, releasing the large, stable HA-carboxylate leaving group and reforming ethylene sulfide.

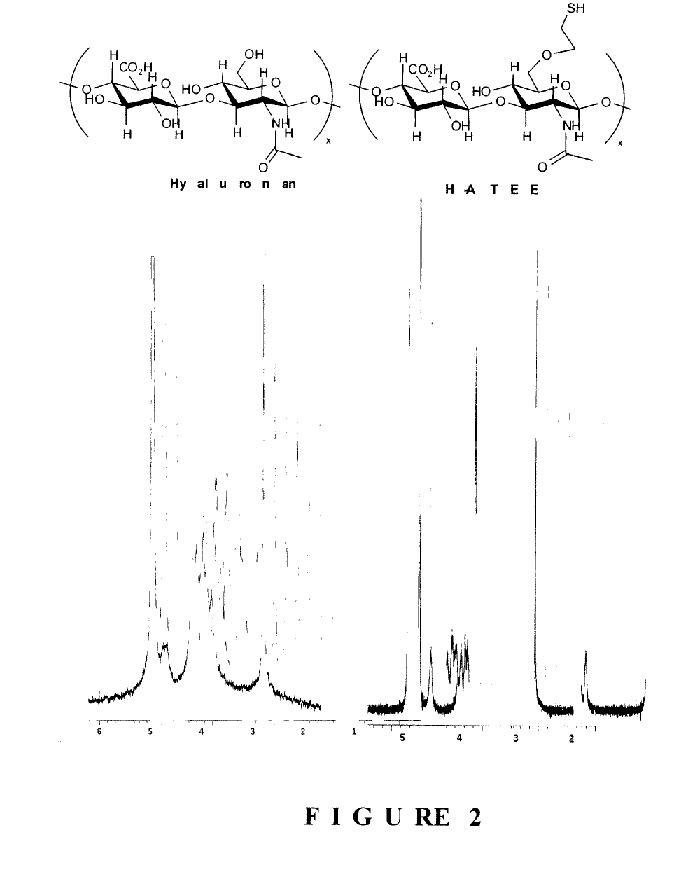

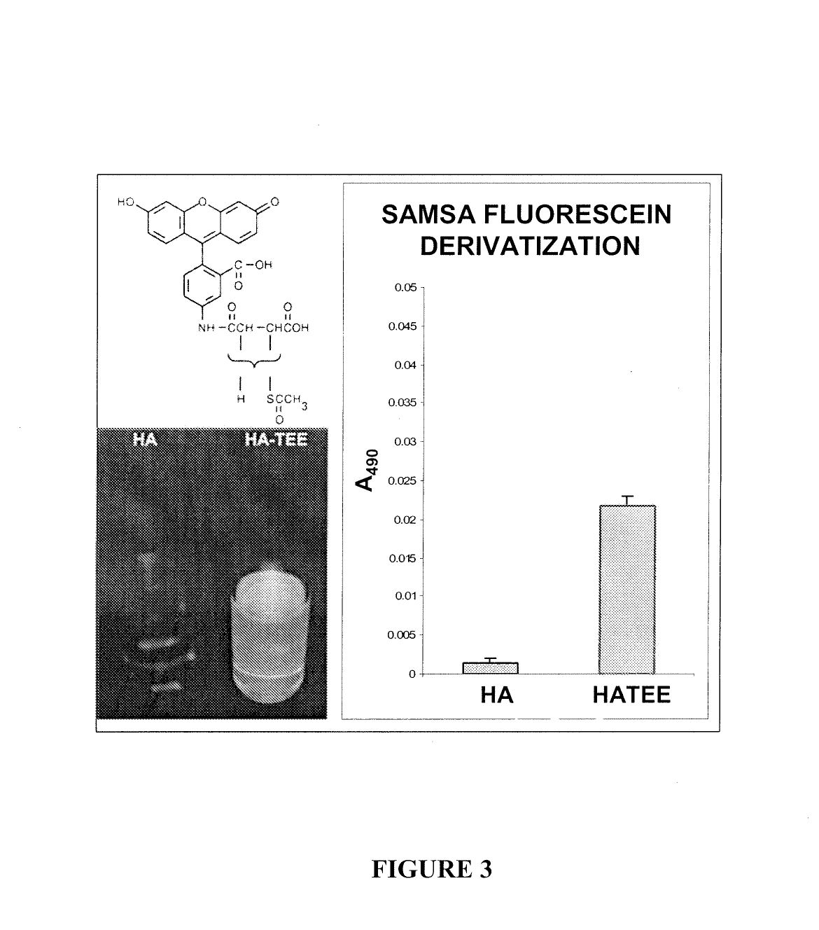

The structure of the new compound was verified by ¹H-NMR (FIG. 2). When compared to ¹H-NMR spectrum of HA (FIG. 2A), a peak corresponding to the methylene group attached to the former hydroxyl oxygen (—CH₂—CH₂—SH), appeared at δ=3.82 ppm. The resonance for the second methylene group, closer to the thiol functionality (—CH₂—CH₂—SH) appears at δ=3.69, but is overlapping with proton resonances corresponding to GlcA and GlcNAc protons from the 3-4 ppm region (FIG. 2B). The integration of the methylene proton signals relative to the N-acetyl protons of GlcNAc could not be used to determine the degree of HA substitution due to the overlapping of the signals. Thus, a modified Ellman's spectroscopic method was employed. The degree of thiolation was determined to be 7-14%. The purity and the molecular weight of HASH (MW˜180 kDa) were determined by GPC analysis (FIG. 3).

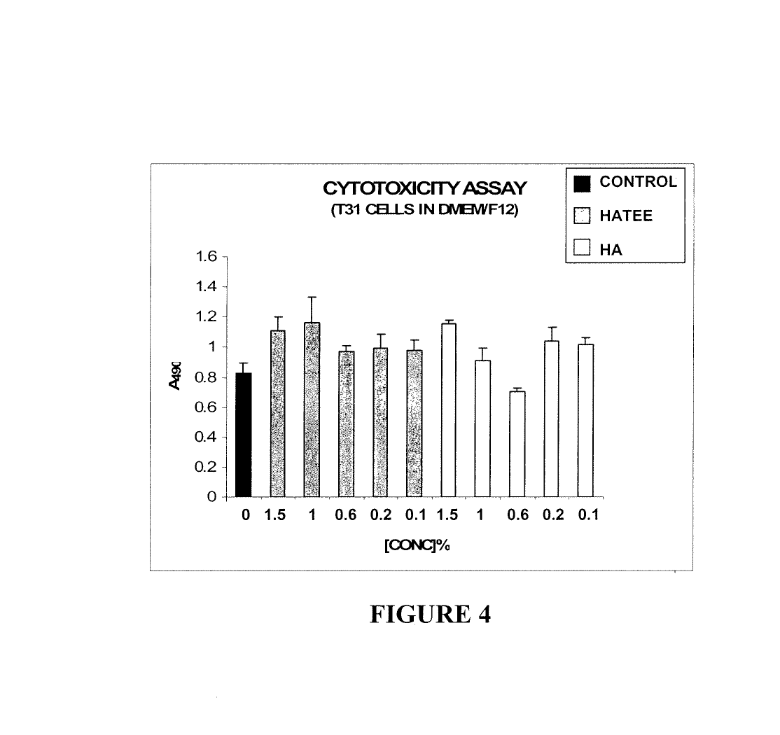

Confirmation of Thiol Modification. Due to the complexity of the polymer proton ¹H-NMR spectra, three additional measures to demonstrate the desired chemical modification were employed. First, we used SAMSA fluorescein, a thiol group-containing fluorescent reagent, commonly used for assaying thiol-reactive maleimide and iodoacetamide moieties of proteins (FIG. 4A), but also suitable for conducting a thiol-disulfide exchange reaction. Due to the ease of monitoring, this molecule was chosen to assess the presence and reactivity of the SH moieties of HASH. After conjugation of HA and HASH with SAMSA fluorescein and dialysis, the solutions were photographed under UV light (254 nm) to assess the fluorescence intensities (FIG. 4, inset). The 412 nm absorbance values of the derivatized compounds were examined, showing that addition of the fluorescent dye to the new moieties occurred (FIG. 4B).

Second, we examined the reaction of a standard thiol-reactive reagent, 4-(hydroxymercuri)benzoic acid sodium salt, with HASH (FIG. 5). This compound was selected because of the downfield aromatic proton resonances would provide well-resolved, sharp, characteristic signature peaks in the NMR, and took advantage of the high affinity and specificity of organomercury reagents for thiols. Upon completion of the reaction and removal of the unreacted, precipitated reagent, the conjugated HASH compound was analyzed by ¹H-NMR (FIG. 5). The two methylene protons of the thiol substituent (—CH₂—CH₂—SH) shifted upfield to the δ=3-3.7 ppm region) and the resonances corresponding to the benzoic acid moiety (—C₆H₄—) appeared at δ=7.4 and δ=7.7 ppm.

Third, we examined the reaction of HASH with sodium iodoacetate (FIG. 6), a reagent commonly employed for “capping” cysteine residues of proteins prior to Edman degradation or proteolysis. As expected, this also resulted in an upfield shift of the methylene protons (—CH₂—CH₂—SH) (δ=3-3.7 ppm region). Altogether, these three reactions confirmed the presence of the thiol modification.

Attempted Crosslinking. The crosslinking of HASH was investigated with a wide spectrum of bivalent electrophilic crosslinkers was evaluated (Table 1), as well as oxidative crosslinking in air and using dilute hydrogen peroxide. To confirm the reactivity of the crosslinkers and to verify the optimal pH for gelation, thiol-derivatized carboxymethylated HA (CMHA-S) was used as positive control in all crosslinking experiments. As anticipated, the control CMHA-S solutions gelled in times ranging from 5 see to 2 h, depending on the nature of the crosslinker and the pH of the solution (Table 2). Next, two different HASH concentrations were used, and the HASH:crosslinker molar ratios ranging from 1:3 to 5:1 were evaluated. Surprisingly, no crosslinking was observed for HASH regardless of the pH of the solution, nature or ratio of crosslinker (Table 3).

TABLE 2
Crosslinking of CMHA-S with bivalent electrophiles as a positive
control for efforts to crosslink HASH. A test tube inversion method
was used to determine the gelation rates and optimal pH values.

| | Molar ratio, | Crosslinking | Optimum |
|---|---|---|---|
| Crosslinker | CMHA-S:crosslinker | time (min) | pH |
| PEGDA | 1:1 | 20 | 7-8 |
| PEGDBrAc | 1:1 | 3.5 | 9-10 |
| PEGDIAc | 1:1 | 0.25 | 9-10 |
| PEGDMal | 1:1 | 0.09 | 7-8 |
| HABA | 3:1 | 120 | 9-10 |
| HAIA | 3:1 | 120 | 9-10 |
| H₂O₂ | 1:1 | 10 | 7-8 |

TABLE 3
Attempted crosslinking of HASH solutions with electrophiles
employed in Table 2. None of the solutions gelled, as indicated
by the “∞” symbol in each entry.
Time for crosslinking at pH 6-pH 10

| Molar ratio, | | | | | | | |
|---|---|---|---|---|---|---|---|
| HASH:Crosslinker | PEGDA | PEGDBrAc | PEGDIAc | PEGDMal | HABA | HAIA | H₂O₂ |
| 1:1 | ∞ | ∞ | ∞ | ∞ | ∞ | ∞ | ∞ |
| 1:2 | ∞ | ∞ | ∞ | ∞ | ∞ | ∞ | ∞ |
| 1:3 | ∞ | ∞ | ∞ | ∞ | ∞ | ∞ | ∞ |
| 2:1 | ∞ | ∞ | ∞ | ∞ | ∞ | ∞ | ∞ |
| 3:1 | ∞ | ∞ | ∞ | ∞ | ∞ | ∞ | ∞ |
| 4:1 | ∞ | ∞ | ∞ | ∞ | ∞ | ∞ | ∞ |
| 5:1 | ∞ | ∞ | ∞ | ∞ | ∞ | ∞ | ∞ |

HASH features reactive thiols that were readily alkylated by the monovalent thiol reagents iodoacetate and p-hydroxymercuribenzoate, and underwent a thiol-disulfide exchange reaction with SAMSA-fluorescein. Thus, the inability to crosslink this polymeric polythiol was unexpected. Three explanations are plausible. First, the low degree of derivatization (7-14% in HASH versus 35-40% in CMHA-S) may be partially responsible for the inability to form a HASH hydrogel. However, we have observed that even 15% thiolation is adequate for gelation of CMHA-S. Second, the 2-thioethyl ether reaches only three atoms (—C—C—S) beyond the primary 6-hydroxyl group, in contrast to the seven (—C—C(O)—N—N—C—C—S) atom extension beyond the same OH group in CMHA-S. This would lead to significantly greater steric hinderance by the bulky HA scaffold, thus impeding access of a single bivalent crosslinker to two separate thioethyl ether sulfhydryl groups. Finally, the reactivity of the 2-thioethyl ether thiol group will be reduced relative to the thiol of the thiopropanoyl hydrazide. We earlier observed significant sensitivity to hydrogel formation between 3-thiopropanoyl hydrazide modified HA and 4-thiobutanoyl hydrazide-modified HA. A difference of only 0.2 pKa units changed gelation rates over 10-fold.

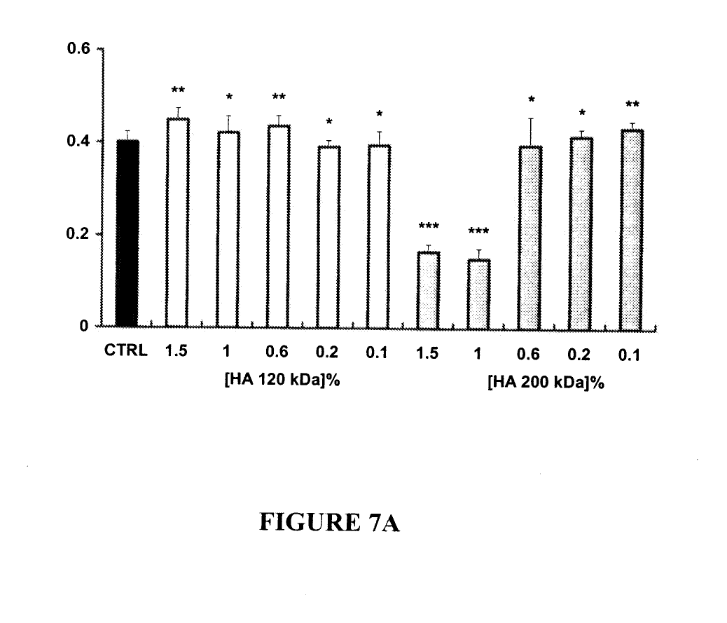

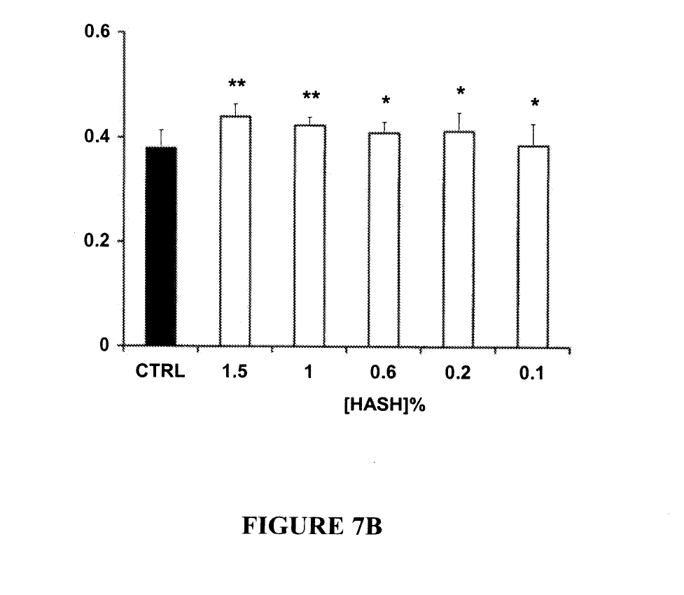

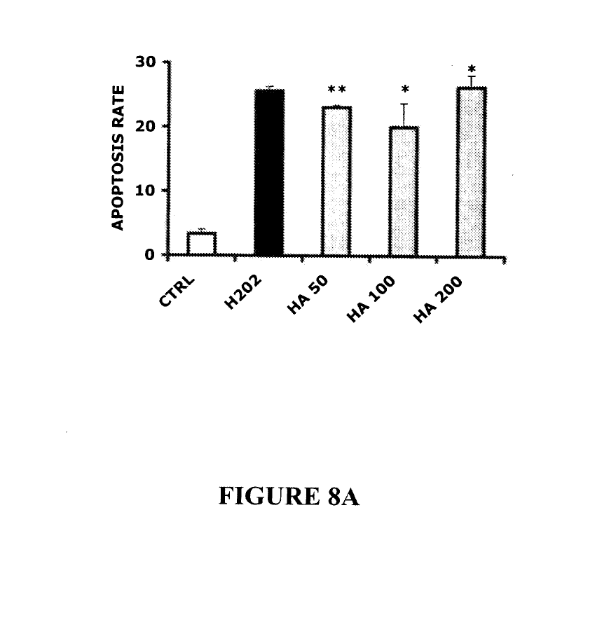

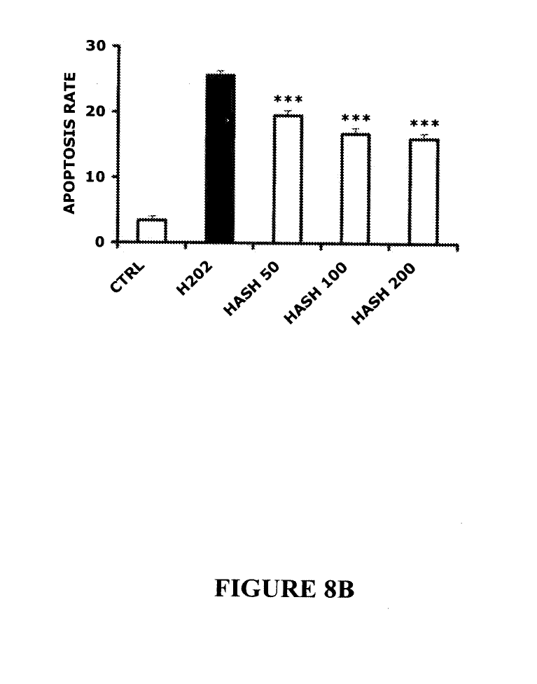

Cytocompatibility of HASH. T31 fibroblasts isolated form human tracheal scar were used to evaluate the cytocompatibility of HASH. These cells are derived from primary culture and were chosen because of their sensitivity to a variety of stressors. For this assay, the newborn calf serum and L-glutamine were excluded from the media to avoid the potential neutralization of HASH (FIG. 7). As controls, two different molecular weight HAs were used (MW 120 and 200 kDa). The 120 kDa HA had no cytotoxic effect on fibroblasts regardless of the concentration used. In contrast, the 200 kDa HA was deleterious (p<0.001) at high concentrations (0.6% to 1.5 w/v) but was well tolerated at low concentrations (0.2-0.1% w/v). This apparent toxicity was due to increased viscosity and the resulting reduction of nutrient diffusibility in the medium. At all concentrations, the effects of HASH were similar to those of the 120 kDa HA on T31 fibroblasts.

Chondroprotective Effects of HASH. Next, we determined the effect of HASH on the apoptosis rates of chondrocytes treated with H₂O₂, and compared the effect of HASH with the effect of unmodified native 120 kDa HA. Samples treated with HA prior to oxidative stress by this surrogate reactive species showed slightly decreased apoptosis rates at 50 μg/mL HA, showing a modest but significant 10% decrease in apoptosis (p<0.05). However, this effect was not dose dependent for unmodified HA, as neither 100 μg/mL nor 200 μg/mL significantly reduced chondrocytes apoptosis (p>0.05). In contrast, HASH protected chondrocytes from reactive oxygen species in a dose dependent manner, with an approximately 40% decrease in the apoptotic rate at the highest concentration (200 μg/mL, p<0.005).

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed:

1. A compound comprising the formula I
Y—X—R—SH I
wherein:
Y is a residue of a macromolecule selected from the group consisting of an oligonucleotide, a nucleic acid or a metabolically stabilized analogue thereof, a polypeptide, a glycoprotein, a glycolipid, a polysaccharide, a protein and a glycosaminoglycan;
X is —O—, —S—, —NH—, or —NR′—;
R′ is C₁₋₅ alkyl; and
R is a substituted or unsubstituted C₂ or C₃ alkylene group.

2. The compound of claim 1, wherein the polysaccharide is selected from the group consisting of hyaluronan, chondroitin sulfate, dermatan, heparan, heparin, dermatan sulfate, heparan sulfate, alginic acid, pectin, chitosan and carboxymethylcellulose.

3. The compound of claim 1, wherein the macromolecule is a protein, selected from the group consisting of a naturally-occurring protein, a recombinant protein, an extracellular matrix protein, a chemically-modified extracellular matrix protein, a partially hydrolyzed derivative of an extracellular matrix protein, and a genetically engineered protein.

4. The compound of claim 1, wherein Y comprises a residue of hyaluronan.

5. The compound of claim 1, wherein Y comprises a residue of a N-acetyl-glucosamine, wherein at least one primary C-6 hydroxyl group of the N-acetyl-glucosamine residue is substituted with the group —RSH.

6. The compound of claim 5, wherein at least one secondary hydroxyl group is substituted with the group —RSH.

7. The compound of claim 6, wherein from one primary C-6 hydroxyl group of the N-acetyl-glucosamine residue to about 100% of the primary C-6 hydroxyl groups of the N-acetyl-glucosamine residue are substituted with the group —RSH.

8. The compound of claim 1, wherein R is selected from the group consisting of CH₂CH₂, CH₂CH₂CH₂, CH₂CHR¹, CHR¹CHR¹, C(R¹)₂CHR¹ and C(R¹)₂C(R¹)₂, wherein R¹ is an alkyl group.

9. The compound of claim 1, wherein R is CH₂CH₂.

10. The compound of claim 1, wherein X is —O— or —NH—.

11. The compound of claim 1, wherein Y is a residue of a hyaluronan, and X is —O—, wherein at least one hydroxyl group is substituted with —CH₂CH₂SH.

12. A method for making a compound, comprising reacting a macromolecule comprising at least one nucleophilic group with a compound comprising the formula XV 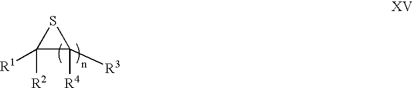
wherein R¹, R², R³, and R⁴ are, independently, hydrogen, an alkyl group, a perfluoroalkyl group, an aryl group, or a heteroaryl group, and n is 1 or 2;
wherein the macromolecule is selected from the group consisting of an oligonucleotide, a nucleic acid or a metabolically stabilized analogue thereof, a polypeptide, a glycoprotein, a glycolipid, a polysaccharide, a protein and a glycosaminoglycan.

13. The method of claim 12, wherein the macromolecule comprises a glycosaminoglycan or a hyaluronan.

14. The method of claim 13, wherein n is 1.

15. The method of claim 13, wherein n is 1 and R¹-R⁴ are hydrogen.

16. The method of claim 13, wherein n is 1, R¹-R⁴ are hydrogen, and the macromolecule is hyaluronan.

17. A method for reducing or preventing inflammation in a subject with inflammation or at risk for inflammation, comprising administering an effective amount of one or more the compounds of claim 1.

18. The method of claim 17, wherein the inflammation is selected from the group consisting of pulmonary inflammation, vascular inflammation, renal inflammation, inflammation of the central nervous system, hepatic inflammation, inflammation in a joint and splanchnic inflammation.

19. The method of claim 18, wherein the compound is administered to the subject systemically, locally, transdermally, or topically.

20. The method of claim 17, wherein the inflammation is associated with an inflammatory disease.

21. The method of claim 20, wherein the inflammatory disease is selected from the group consisting of systemic lupus erythematosus, Hashimoto's disease, rheumatoid arthritis, graft-versus-host disease, Sjogren's syndrome, pernicious anemia, Addison disease, scleroderma, Goodpasture's syndrome, Crohn's disease, autoimmune hemolytic anemia, myasthenia gravis, multiple sclerosis, Alzheimer's disease, amyotrophic lateral sclerosis, Basedow's disease, thrombopenia purpura, insulin-dependent diabetes mellitus, allergy; asthma, inflammatory bowel disease, cancer, ulcerative colitis, scleroderma, cardiomyopathy, atherosclerosis, hypertension, sickle cell disease, and respiratory distress syndrome of neonate and adults.

22. The method of claim 17, wherein the inflammation is caused by an organ transplantation, respiratory distress, ventilator induced lung injury, ischemia reperfusion, hemorrhagic shock, or sepsis.

23. The method of claim 17, wherein when the inflammation is caused by respiratory distress or sepsis, and wherein the compound reduces or prevents the accumulation of alveolar fluid in a subject.

24. A method for reducing or preventing damage to a cell or tissue caused by a free radical or reactive oxygen species, comprising contacting the cell with of one or more compounds of claim 1.

25. The method of claim 24, wherein the reactive oxygen species is selected from the group consisting of NO., HO., HOO.⁻, HOO. and O₂.⁻.

26. The method of claim 24, wherein the free radical or reactive oxygen species is produced by exposure of the cell to radiation.

27. A method for reducing or preventing the formation of scar tissue in a subject produced by a free radical or reactive oxygen species, comprising administering an effective amount of one or more compounds of claim 1.

28. A method for growing cells, comprising contacting the cells with one or more compounds of claim 1.

29. A method for growing tissues, comprising contacting precursor cells with one or more compounds of claim 1.

30. A method for preserving an organ, tissue, or cells comprising contacting the organ, tissue, or cells with a compound of claim 1.

31. A method for protecting an organ, tissue, or cells from exposure to a reactive oxygen species, comprising contacting the organ, tissue, or cells with a compound of claim 1.

32. A method for preventing or reducing ischemic reperfusion in a tissue of a subject, comprising contacting the tissue with a compound of claim 1.

33. A method for coupling two or more thiolated compounds, comprising reacting a first thiolated compound comprising the formula I of claim 1 with a second thiolated compound having at least one SH group in the presence of an oxidant, wherein the first thiolated compound and second thiolated compound are the same or different compounds.

34. The method of claim 33, wherein the second thiolated compound is a macromolecule selected from the group consisting of an oligonucleotide, a nucleic acid or a metabolically stabilized analogue thereof, a polypeptide, a glycoprotein, a glycolipid, or a pharmaceutically-acceptable compound.

35. The method of claim 33, wherein the second thiolated compound comprises a polysaccharide having at least one SH group.

36. The method of claim 33, wherein the second thiolated compound comprises a sulfated-glycosaminoglycan.

37. The method of claim 33, wherein the second thiolated compound is selected from the group consisting of chondroitin sulfate, dermatan, heparan, heparin, dermatan sulfate, heparan sulfate, alginic acid, pectin, chitosan, carboxymethylcellulose and hyaluronic acid having at least one SH group.

38. The method of claim 33, wherein the second thiolated compound comprises a thiolated protein.

39. The method of claim 33, wherein the first thiolated compound and the second thiolated compound are different.

40. The method of claim 33, wherein the oxidant comprises oxygen.

41. The method of claim 40, wherein the oxidant further comprises hydrogen peroxide.

42. A compound prepared by the method of claim 33.

43. A compound having at least one fragment comprising the formula VI 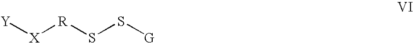
wherein:
Y is a residue of a first macromolecule selected from the group consisting of an oligonucleotide, a nucleic acid or a metabolically stabilized analogue thereof, a polypeptide, a glycoprotein, a glycolipid, a polysaccharide, a protein and a glycosaminoglycan;
X is —O—, —S—, —NH—, or —NR′—;
R′ is hydrogen or C₁₋₅ alkyl;
R is a substituted or unsubstituted C₂ or C₃ alkylene group; and
G is a residue of a second macromolecule selected from the group consisting of an oligonucleotide, a nucleic acid or a metabolically stabilized analogue thereof, a polypeptide, a glycoprotein, a glycolipid, a polysaccharide, a protein and a glycosaminoglycan.

44. The compound of claim 43, wherein the first or the second macromolecule is independently selected from the group consisting of an oligonucleotide, a nucleic acid or a metabolically stabilized analogue thereof, a polypeptide, a glycoprotein, a glycolipid, a polysaccharide, a protein, a synthetic polymer and a glycosaminoglycan.

45. The compound of claim 43, wherein Y is a residue of a macromolecule selected from the group consisting of chondroitin sulfate, dermatan, heparan, heparin, dermatan sulfate, heparan sulfate, alginic acid, pectin, chitosan and carboxymethylcellulose.

46. The compound of claim 43, wherein Y is a residue of a hyaluronan, X is oxygen, and R is —CH₂CH₂—.

47. The compound of claim 43, wherein G comprises a polysaccharide residue.

48. The compound of claim 43, wherein G comprises a glycosaminoglycan residue.

49. The compound of claim 43, wherein G is a residue selected from the group consisting of chondroitin sulfate, dermatan, heparan, heparin, dermatan sulfate, heparan sulfate, alginic acid, pectin, chitosan, carboxymethylcellulose and hyaluronan.

50. A method for making a compound, comprising reacting a first thiolated macromolecule comprising the formula I in claim 1, with at least a second compound having at least one thiol-reactive electrophilic functional group.

51. The method of claim 50, wherein the second compound has at least two thiol-reactive electrophilic groups.

52. The method of claim 50, wherein the second compound has at least two haloacetate groups.

53. The method of claim 50, wherein the macromolecule is selected from the group consisting of an oligonucleotide, a nucleic acid or a metabolically stabilized analogue thereof, a polypeptide, a glycoprotein, a glycolipid, a polysaccharide, a protein, a synthetic polymer and glycosaminoglycan.

54. The method of claim 50, wherein the thiolated macromolecule has the formula Y—X—R—SH, wherein Y is a residue of a hyaluronan, X is oxygen, and R is —CH₂CH₂—.

55. The method of claim 50, further comprising a second thiolated macromolecule, wherein the first and second macromolecule are the same or different.

56. The method of claim 50, wherein the thiol-reactive electrophilic functional group comprises an electron-deficient vinyl group.

57. The method of claim 50, wherein the compound has two electron-deficient vinyl groups, wherein the two electron-deficient vinyl groups are the same.

58. The method of claim 50, wherein the compound comprises a diacrylate, a dimethacrylate, a diacrylamide, a dimethacrylamide, a vinyl sulfone, a maleimide, or a combination thereof.

59. The method of claim 50, wherein the second compound has the formula V 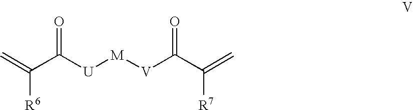
wherein
R⁶ and R⁷ are, independently, hydrogen or lower alkyl;
U and V are, independently, —O— or —NR⁸— wherein each R⁸ is, independently, hydrogen or lower alkyl; and
M is selected from the group consisting of a polyalkylene group, a polyether group, a polyamide group, a polyimino group, a polyester, an aryl group, and a polythioether group.

60. The method of claim 50, wherein the compound comprises the formula XX 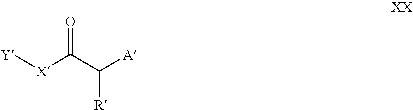
wherein
Y′ is a residue of a macromolecule;
X′ is —O—, —S—, —NH— or —NR″—;
R′ is hydrogen, an alkyl group, a perfluoroalkyl group, an aryl group, a heteroaryl group, or a halogen;
R″ is hydrogen or C₁₋₅ alkyl; and
A′ is a leaving group.

61. The method of claim 60, wherein Y′ comprises a residue of hyaluronan.

62. The method of claim 60, wherein Y′ comprises a residue of a N-acetyl-glucosamine, wherein at least one primary C-6 hydroxyl group of the N-acetyl-glucosamine residue is substituted with the group —C(O)CH(R′)(A′).

63. The method of claim 62, wherein at least one secondary hydroxyl group of the glucosamine is substituted with the group —C(O)CH(R′)(A′).

64. The method of claim 62, wherein from one primary C-6 hydroxyl group of the N-acetyl-glucosamine residue to about 100% of the primary C-6 hydroxyl groups of the N-acetyl-glucosamine residue are substituted with the group —C(O)CH(R)(A′).

65. The method of claim 60, wherein R′ is methyl or hydrogen.

66. The method of claim 60, wherein A′ comprises a halogen.

67. The method of claim 60, wherein Y′ is a residue of a hyaluronan, wherein at least one hydroxyl group of hyaluronan is substituted with —C(O)CH₂Cl, —C(O)CH₂Br, or —C(O)CH₂I.

68. A compound produced by the method of claim 50.

69. A compound having at least one fragment comprising the formula VII 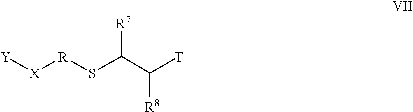
wherein:
R⁷ and R⁸ are, independently, hydrogen or lower alkyl;
T is an electron-withdrawing group;
Y is a residue of a macromolecule;
X is —O—, —S—, —NH—, or —NR′—;
R′ is hydrogen or C₁₋₅ alkyl; and
R is a substituted or unsubstituted C₂ or C₃ alkylene group.

70. A pharmaceutical composition comprising a pharmaceutically-acceptable compound comprising the compound of claim 1.

71. A pharmaceutical composition comprising a pharmaceutically-acceptable compound comprising the compound of claim 43.

72. A pharmaceutical composition comprising a living cell and the compound of claim 1.

73. A pharmaceutical composition comprising a living cell and the compound of claim 43.

74. A method for improving wound healing in a subject in need of such improvement, comprising contacting the wound of the subject with the compound of claim 1.

75. A method for improving wound healing in a subject in need of such improvement, comprising contacting the wound of the subject with the compound of claim 43.

76. A method for delivering at least one pharmaceutically-acceptable compound to a patient in need of such delivery, comprising contacting at least one tissue capable of receiving the pharmaceutically-acceptable compound with the composition of claim 72.

77. The use of the compound of claim 1 as a growth factor, an anti-cancer agent, an analgesic, an anti-infection agent, or an anti-cell attachment agent.

78. The use of the compound of claim 43 as a growth factor, an anti-cancer agent, an analgesic, an anti-infection agent, or an anti-cell attachment agent.

79. The use of the compound of claim 1 in combination with a growth factor, an anti-cancer agent, an analgesic, an anti-infection agent, or an anti-cell attachment agent.

80. The use of the compound of claim 43 in combination with a growth factor, an anti-cancer agent, an analgesic, an anti-infection agent, or an anti-cell attachment agent.

81. A method for reducing or preventing inflammation in a subject with inflammation or at risk for inflammation, comprising administering an effective amount of one or more compounds of claim 43.

82. A method for protecting an organ, tissue, or cells from exposure to a reactive oxygen species, comprising contacting the organ, tissue, or cells with a compound of claim 43.

83. A method for preventing or reducing ischemic reperfusion in a tissue of a subject, comprising contacting the tissue with a compound of claim 43.

84. A composition comprising one or more compounds of claim 1 and water, wherein the compound does not form a hydrogel.

85. A composition comprising one or more compounds of claim 43 and water, wherein the compound does not form a hydrogel.

86. The use of a compound of claim 1 to prevent adhesion after a surgical procedure, wherein the surgical procedure comprises cardiosurgery and articular surgery, abdominal surgery, a surgical procedure performed in the urogenital region, a surgical procedure involving a tendon, ligament, rotator cuff, laparascopic surgery, pelvic surgery, oncological surgery, sinus and craniofacial surgery, ENT surgery, a procedure involving spinal dura repair, or for vocal fold repair, prophylaxis, or restoration of function.

87. The use of a compound of claim 43 to prevent adhesion after a surgical procedure, wherein the surgical procedure comprises cardiosurgery and articular surgery, abdominal surgery, a surgical procedure performed in the urogenital region, a surgical procedure involving a tendon, ligament, rotator cuff, laparascopic surgery, pelvic surgery, oncological surgery, sinus and craniofacial surgery, ENT surgery, a procedure involving spinal dura repair, or for vocal fold repair, prophylaxis, or restoration of function.
